(19) United States
(12) Patent Application Publication
(10) Pub. No.: US 2013/0295060 A1
(43) Pub. Date: Nov. 7, 2013

(54) METHOD FOR CULTURING CARDIAC PROGENITOR CELLS AND USE OF CARDIAC PROGENITOR CELLS

(71) Applicants: Inje University Industry-Academic Cooperation Foundation, (US); Ewha University-Industry Collaboration Foundation, (US)

(72) Inventors: Young Il YANG, Busan (KR); Seung Jin Lee, Seoul (KR); Hyeong In Kim, Busan (KR); Jong Tae Kim, Busan (KR); Soon Ho Cheong, Busan (KR)

(73) Assignees: Ewha University-Industry Collaboration Foundation, Seoul (KR); INJE UNIVERSITY INDUSTRY-ACADEMIC COOPERATION FOUNDATION, Gimhae-si (KR)

(21) Appl. No.: 13/886,663

(22) Filed: May 3, 2013

(30) Foreign Application Priority Data
May 4, 2012 (KR) 10-2012-0047701

(51) Int. Cl. A61K 35/34

(52) U.S. Cl. CPC A61K 35/34
USPC 424/93.7; 435/325; 435/377

(57) ABSTRACT

Disclosed is a method for culturing myocardium-resident cardiac progenitor cells, comprising: embedding myocardial fragments into hydrogel; culturing the myocardial fragment into hydrogel; degrading only the hydrogel to recover cardiac progenitor cells grown out of the myocardial fragment to the hydrogel; and amplifying the cardiac progenitor cells in vitro. Also, the cardiac progenitor cells, a method for differentiating the same, and the use thereof as cell therapeutic agent for heart diseases are provided. In addition to possessing the potential to differentiate into cardiomyocytes, osteoblasts, adipocytes, chondrocytes, vascular endothelial cells, smooth muscle cells, neural cells, and skeletal muscle cells, the myocardium-resident cardiac progenitor cells can spontaneously differentiate into cardiomyocytes even in the absence of a special differentiation inducing agent. Thus, the cardiac progenitor cells can be used to produce bio-active medicines such as cell therapeutics and tissue engineering therapeutics with high industrial applicability.

### BACKGROUND OF THE INVENTION

1. Field of the Invention

The present invention relates to cardiac progenitor cells, a method for culturing the same, a method for differentiating the same, a cell therapeutic agent comprising the same, and a therapeutic agent for heart diseases comprising the same.

2. Description of the Related Art

When it is damaged by various factors, the heart is regenerated, although limitedly. Ischemic heart diseases such as coronary artery disease and myocardial infarction cause death and irreversible loss of cardiomyocytes, resulting in a decrease in heart function, with the consequent onset of intractable myocardial diseases such as congestive heart failure. Hence, there is a pressing need for a novel stem cell-based bio-active medicine that is clinically applicable to such intractable heart diseases.

Stem cell therapeutics applicable to the therapy of intractable myocardial diseases may be sourced from embryonal stem cells (ESCs), induced pluripotent stem cells (iPSCs), or adult stem cells. Both ESCs and iPSCs have the potential to differentiate into all cardiogenic lineages, that is, cardiomyocytes (CMCs), vascular smooth muscle cells (vSMCs), and endothelial cells (ECs), but present difficult technical problems that must be solved before clinical application, such as immune rejection, tumorigenesis, and control of differentiation into cardiac muscle tissues. In contrast, adult stem cells are relatively free of the risk of immune rejection and tumorigenesis. In practice, almost all of the cell therapeutics that are currently applied in the therapy of intractable myocardial diseases are based on adult stem cells.

Among adult stem cells for use in the therapy of intractable myocardial diseases are (1) hematopoietic stem cells, (2) bone marrow- or umbilical cord blood-derived mesenchymal stem cells, (3) skeletal muscle-derived mesenchymal stem cells or skeletal muscle progenitor cells, (4) adipose-derived mesenchymal stem cells, and (5) recently discovered endogenous cardiac progenitor cells (CPCs). Hematopoietic stem cells may be obtained from bone marrow by aspiration. They may also be collected from peripheral blood following pre-treatment with GM-CSF, which induces cells to be mobilized from the bone marrow compartment. Another source of hematopoietic stem cells is umbilical cord blood. However, hematopoietic stem cells are not easy to prepare sufficient therapeutic dose because of difficulty in in vitro proliferation. In addition, hematopoietic stem cells lack the ability to directly differentiate into myocardial cells. Mesenchymal stem cells derived from bone marrow, umbilical cord blood, skeletal muscle, and adipose tissue have an advantage over hematopoietic stem cells in terms of applicability as cell therapeutics thanks to how easily they can undergo in vitro amplification, but they are poor in biological effectiveness as a therapeutic for intractable myocardial diseases due to their lack of ability to directly differentiate into myocardial cells. In contrast, cardiac progenitor cells are the only adult stem cells that are capable of differentiating into all constituent cells of heart. Further, they can be cultured in vitro at high efficiency. Therefore, intensive attention is now being paid to cardiac progenitor cells because they are considered to meet all the requirements for a therapeutic for intractable myocardial diseases.

Typically, cardiac progenitor cells are isolated and cultured using one of the following three methods. A first method starts with a tissue dissociation process in which single cell groups are dissociated from solid heart muscles by treatment with enzymes such as collagenase, dispase, and trypsin, after which cells expressing specific markers are isolated from the dissociated single cell groups and then amplified using a monolayer culture method. In a second method, heart muscles are loosened by mildly enzymatic treatment, seeded to a culture vessel, and cultured in a two-dimensional manner. The final method is characterized by selectively isolating and monolayer culturing cardiospheres which start to form from seven days after the two-dimensional culturing of the second method.

In the first method, c-Kit or Sca-1 is representative of the markers used to isolate cardiac progenitor cells from dissociated single cells. However, these markers are expressed in other stem cells including mesenchymal stem cells and hemapoietic stem cells, besides cardiac progenitor cells. Further, cardiac progenitor cells devoid of these markers undoubtedly exist. Thus, the markers are not improper for use in isolating cardiac progenitor cells. Since no markers specific solely for cardiac progenitor cells have been identified thus far, the immunological isolation of cardiac progenitor cells by using specific markers is always limited. In addition, the immunological isolation of cardiac progenitor cells is necessarily accompanied by the enzymatic treatment of tissues for separating single cells. The quantity of single cells during the tissue dissociation process varies greatly depending on various factors including the kind of enzymes used, titer, reaction time, reaction temperature, and the state of tissues used. Moreover, the cells cannot be prevented from being damaged during the tissue dissociation process. As a result, the tissue dissociation method has the disadvantage of being difficult to standardize, and of being inefficient due to low isolation yield.

In the second method, stable contact between myocardial fragments and a culture vessel into which the myocardial fragments are seeded is one of the factors that plays a critical role in the successful isolation and culture of cardiac progenitor cells. Upon enzymatic treatment to produce myocardial fragments, extracellular matrixes and nucleic acids are also released, and interfere with the stable contact of the myocardial fragments with the culture vessel. Thus, the substrata in which cells derived from the cardiac muscles can adhere and grow are not stably provided, resulting in the inhibition of the stable migration and growth of cardiac progenitor cells. Moreover, a limited surface area of the substrata to which the seeded myocardial fragments are attached imparts a limitation to the migration and growth of cardiac progenitor cells in culture vessels.

The third method is to isolate cardiac progenitor cells from cardiospheres. In this regard, myocardial fragments are seeded in a two-dimensional arrangement into a culture vessel and incubated for seven days as in the second method, after which time small, spherical moving cells are separated and cultured in a serum-free medium to obtain cardiospheres. These cardiospheres are grown in a monolayer culture manner in a culture medium supplemented with serum to form cardiac progenitor cells morphologically similar to fibroblasts. However, this method also has the disadvantages raised by the two-dimensional seeding culture of myocardial fragments, and the complex multi-step procedure lowers the efficiency of culture.

Meanwhile, the excavation of a mechanism and a factor responsible for controlling differentiation into cardiomyocytes is an important subject in order to understand the etiology of intractable heart diseases and to develop a therapeutic for the diseases. So far, there have been very few in vitro experimental models that are useful for understanding the etiology of heart diseases and for developing therapeutics therefor. As models capable of safely and effectively inducing embryonic and adult stem cells or cardiac progenitor cells to differentiate into cardiomyocytes, only the monolayer culture method has been suggested. However, the conventional method suffers from the disadvantage of requiring two or more weeks for the completion thereof and of inducing differentiation at limited efficiency.

In the body, all organs and cells take three-dimensional structures. Cells perform their intrinsic biological functions through interaction between cells, and between cells and extracellular matrixes. Conventional methods cannot guarantee interactions between cells, or between cells and extracellular matrixes, which results in a failure to effectively and stably induce differentiation into cardiomyocytes. Accordingly, an in vitro culture method of providing an environment mimic to practical microarchitecture where cells resided in the body is essential for the development of a therapeutic for intractable heart diseases.

Extensive pre-clinical research into therapeutic effects of stem cells has been made on animal models of cardiovascular diseases, and clinical trials have been applied to myocardial infarction patients. There are antithetic research results with regard to the stability and effectiveness of cell therapeutics. These antithetic research results may be attributed to a difference in the delivery of cell therapeutics into the heart. Of the cells injected through blood vessels, only 0.01% of the cells were known to be migrated to injured myocardium. Upon direct injection of cell therapeutics into cardiac muscles, less than 1% of the cells injected were observed to retain and survive in the injured myocardium. The remaining 99% of the cells were lost or dead by hemorrhage upon injection or by excessive inflammation within injured myocardium. Hence, the stable delivery of cell therapeutics into injured myocardium and the protection of delivered cell therapeutics from hemorrhage and excessive inflammation are significant challenges to be overcome.

In the present invention, stable and effective isolation and incubation of cardiac progenitor cells from adults, without using tissue degrading enzymes or destroying the myocardial microarchitectures where stem cells reside, is provided, together with a method for inducing the differentiation of the cultured cardiac progenitor cells into cardiomyocytes, and a method for delivering them to injured myocardium.

### SUMMARY OF THE INVENTION

It is therefore an object of the present invention to provide a method for isolating and culturing myocardium-resident cardiac progenitor cells stably and effectively, without tissue dissociation.

It is another object of the present invention to provide myocardium-resident cardiac progenitor cells, cultured using the method, which possess a potential for differentiation into multiple cardiogenic lineages and robust ex vivo expandability.

It is a further object of the present invention to provide a method for the differentiation of myocardium-resident cardiac progenitor cells into cardiomyocytes, using a suspension cell culture condition.

It is still another object of the present invention to provide a cell therapeutic agent or a pharmaceutical composition for the prophylaxis or therapy of heart diseases, comprising cardiac progenitor cells or cells differentiated therefrom as an active ingredient.

In accordance with an aspect thereof, the present invention provides a method for culturing myocardium-resident cardiac progenitor cells, comprising: embeddig myocardial fragments into hydrogel; culturing the myocardial fragment embedded into hydrogel; degrading only the hydrogel to recover cardiac progenitor cells grown out of the myocardial fragment to the hydrogel; and amplifying the cardiac progenitor cells in vitro.

In one embodiment, the cardiac progenitor cells obtained using the method exhibit at least one immunological trait of: (i) being positive to a cardiac progenitor cell marker selected from the group consisting of nestin, Sca-1, and a combination thereof; (ii) being positive to a cardiomyocyte-specific transcription factor marker selected from the group consisting of GATA-4, Nkx-2.5, Mef-2c, and a combination thereof; (iii) being positive to a mesenchymal stem cell marker selected from the group consisting of CD29, CD44, CD73, CD90, CD105, and a combination thereof; (iv) being positive to a vascular pericyte marker selected from the group consisting of CD140b, CD146, α-smooth muscle actin (SMA), and a combination thereof; (v) being negative to a hematopoietic cell marker selected from Lin, CD34, CD45, and a combination thereof; (vi) being negative to a vascular endothelial cell marker selected from the group consisting of CD31, CD34, and a combination thereof; and (vii) being negative to a cardiomyocyte marker selected from the group consisting of α-sarcometric actinin (α-SA), myosin heavy chain (MHC), troponin I (TnI), troponin T (TnT), and a combination thereof.

In another embodiment, the hydrogel is not particularly limited with regard to its kinds, and may be made of a natural polymer.

In another embodiment, the hydrogel may comprise an antifibrinolytic agent.

No particular limitations are imparted with regard to kinds of the antifibrinolytic agent. Examples of the antifibrinolytic agent include aminocaproid acid, tranexamic acid, aprotinin, aminomethylbenzoic acid, and a combination thereof.

In another embodiment, the antifibrinolytic agent may be used in an amount of from 10 to 1000 μg in 1 ml of the hydrogel.

The hydrogel may be a fibrin hydrogel containing fibrinogen in a concentration of from 0.8 to 5.0 mg/ml.

In another embodiment, the hydrogel may be degraded by an enzyme selected from the group consisting of collagenase, gelatinase, urokinase, streptokinase, tissue plasminogen activator (TPA), plasmin, hyaluronidase, and a combination thereof.

In another embodiment of the method of the present invention, 1) the embedding is carried out by mixing the myocardial fragment with a fibrin hydrogel containing an antifibrinolytic agent selected from the group consisting of aminocaproid acid, tranexamic acid, and a combination thereof; 2) the culturing is carried out by subjecting the myocardial fragment embedded into the fibrin hydrogel to a three-dimensional organ culture while shaking at 5 to 30 rpm to allow myocardial-resident cardiac progenitor cells to grow out of the myocardial fragment to the hydrogel; 3) the degrading is carried out by treating the fibrin hydrogel with an enzyme selected from the group consisting of urokinase, streptokinase, plasmin, and a combination thereof to recover the myocardium-resident cardiac progenitor cells and the myocardial fragment; and 4) the amplifying is carried out by culturing the cardiac progenitor cells recovered from the hydrogel in a monolayer culture condition.

In another embodiment, the recovered myocardial fragment may be recycled by being embedded again into a hydrogel.

In accordance with another aspect thereof, the present invention provides cardiac progenitor cells, obtained using the culturing method, exhibiting at least one immunological trait of: (i) being positive to a cardiac progenitor cell marker selected from the group consisting of nestin, Sca-1 and a combination thereof; (ii) being positive to cardiomyocyte-specific transcription factor marker selected from the group consisting of GATA-4, Nkx-2.5, Mef-2c, and a combination thereof; (iii) being positive to a mesenchymal stem cell marker selected from the group consisting of CD29, CD44, CD73, CD90, CD105, and a combination thereof; (iv) being positive to a vascular pericyte marker selected from the group consisting of CD140b, CD146, α-smooth muscle actin (SMA), and a combination thereof; (v) being negative to a hematopoietic cell marker selected from Lin, CD34, CD45, and a combination thereof; (vi) being negative to a vascular endothelial cell marker selected from the group consisting of CD31, CD34, and a combination thereof; and (vii) being negative to a cardiomyocyte marker selected from the group consisting of α-sarcometric actinin (α-SA), myosin heavy chain (MHC), troponin I (TnI), troponin T (TnT), and a combination thereof.

In accordance with a further aspect thereof, the present invention provides a method for differentiating myocardium-resident cardiac progenitor cells, comprising culturing the cardiac progenitor cells in a suspension cell culture condition.

In one embodiment, the cardiac progenitor cells are capable of spontaneously differentiating into cardiomyocytes.

In accordance with still another aspect thereof, the present invention provides a cell therapeutic agent, comprising the cardiac progenitor cells, or cells differentiated therefrom, as an active ingredient.

In one embodiment, the cell therapeutic agent may treat cells selected from the group consisting of cardiomyocytes, osteoblasts, adipocytes, chondrocytes, vascular endothelial cells, smooth muscle cells, neural cells, skeletal muscle cells, and a combination thereof.

In another embodiment, the cardiac progenitor cells are in mixture with a hydrogel containing an antifibrinolytic agent.

In another embodiment, the cell therapeutic may further comprise a factor selected from the group consisting of an anti-inflammatory agent, a stem cell-mobilizing factor, a vascular growth inducing factor, and a combination thereof.

In accordance with a still further aspect thereof, the present invention provides a pharmaceutical composition for prophylaxis or therapy of a heart disease, comprising the cardiac progenitor cells or cells differentiated therefrom as an active ingredient, wherein the progenitor cells are in mixture with a hydrogel containing an antifibrolytic agent.

In one embodiment, the heart disease may be selected from the group consisting of myocardial infarction, ischemic myocardial disease, primary myocardial disease, secondary myocardial disease, congestive heart failure, and a combination thereof.

In addition to having the potential to differentiate into all cardiogenic lineages, the myocardium-resident cardiac progenitor cells of the present invention can be proliferated in vitro in a high yield. Further, the cardiac progenitor cells can spontaneously differentiate into cardiomyocytes even in the absence of a special differentiation inducing agent, and can survive in vivo with great efficiency after transplantation. Thanks to these advantages, the cardiac progenitor cells can be used to produce bio-active medicines such as cell therapeutics and tissue engineering therapeutics, with high industrial applicability. In addition, the cardiac progenitor cells of the present invention can find applications in various fields relevant to the mobilization, migration, growth, and differentiation of cardiac progenitor cells, including cell biological and molecular biological research and novel medicine development.

### BRIEF DESCRIPTION OF THE DRAWINGS

The above and other objects, features and other advantages of the present invention will be more clearly understood from the following detailed description taken in conjunction with the accompanying drawings, in which:

FIG. 1 shows the microarchitectures of fibrin according to the concentration of fibrinogen and to the presence of plasminogen activator inhibitor (AMBA) in fluorescence photographs (A), and the pore sizes of fibrin according to the concentration of fibrinogen in a graph (B) (*, p<0.01 in comparison with 0.5% and 1.0% fibrinogen);

FIG. 2 is a graph showing the fibrinolytic effect of cardiac progenitor cells. Fibrin composed by varying concentrations of the fibrinogen was degraded by the cardiac progenitor cells, which are embedded into and three-dimensionally cultured in four types of fibrin hydrogels prepared from 1.25, 2.5, 5.0, or 10.0 mg/ml fibrinogen and 0.5 units/ml thrombin (*, p<0.01 compared to w/o CPCs);

FIG. 3 is a graph the inhibitory effects of antifibrolytic agents on cardiac progenitor cell-induced fibrinolysis (*, p<0.01 compared to aprotinin and aminocaproid acid);

FIG. 4 shows photographs of fibrin hydrogels which are used as three-dimensional substrate for cardiac progenitor cells. Fibrin hydrogel maintains their structure against the fibrinolysis of cardiac progenitor cells when containing aminomethylbenzoic acid;

FIG. 5 shows the cytoplasmic spreading of cardiac progenitor cells in hydrogels containing aminomethylbenzoic acid in a phase-contrast microphotograph (upper panels, A), a fluorescence microphotograph (lower panels, A), and a bar graph (B) where the concentration of fibrinogen in the hydrogel reduces the degree of cytoplasmic spreading in a dose-dependent manner (*, p<0.01 compared to 1.25 and 2.5 mg/ml);

FIG. 6 is a graph showing the growth of cardiac progenitor cells in aminomethylbenzoic acid-containing hydrogels in terms of DNA content in which fibrinogen reduces DNA contents in a dose-dependent manner (*, p<0.01 compared to 1.25 and 2.5 mg/ml fibrinogen);

FIG. 7 shows phase-contrast microphotographs of three-dimensional organ cultures of myocardial fragments in a hydrogel void of antifibrinolytic agents or containing an antifibrinolytic agent (A) where the antifibrinolytic agent-void hydrogel cannot serve as a three-dimensional cell adhesion matrix due to the fibrinolytic activity of the myocardial fragments, thus incapacitating the migration and growth of cardiac progenitor cells (No, white arrows), whereas the hydrogel containing tranexamic acid or aminomethylbenzoic acid serves as a cell adhesion matrix in which the cardiac progenitor cells from the myocardial fragments grow, and a bar graph (B) in which the migration and growth of the cells from the myocardial fragments is quantitatively plotted against the concentration of fibrinogen in an antifibrinolytic agent-containing hydrogel (*, p<0.01 compared to 1.25 and 2.5 mg/ml fibrinogen);

FIG. 8 is a schematic diagram illustrating a three-dimensional organ culture of myocardial fragments in an antifibrinolytic agent-containing hydrogel, and the isolation of cardiac progenitor cells through the organ culture;

FIG. 9 shows phase-contrast microphotographs (upper panels) and immunochemistry staining photographs (lower panels) of cells grown out of myocardial fragments in hydrogel after the three-dimensional organ culture of human myocardial fragments is placed in the hydrogel;

FIG. 10 shows activities of integrin-mediated signaling pathway factors within myocardial fragments before organ culture (Fresh) and after organ culture of the myocardial fragments without a support of hydrogel (2D) and three-dimensional organ culture of the myocardial fragments in an antifibrolytic agent-containing hydrogel (3D) in which the hydrogel is proven to increase the activities of the integrin-mediated signaling pathway factors (*, p<0.05 compared to ‘Fresh’; #, p<0.05 compared to ‘Fresh’ and ‘2D’);

FIG. 11 is a graph showing the effect of dynamic culture conditions on the migration and growth of cardiac progenitor cells from mouse, rat, and human myocardial fragments embedded into an antifibrinolytic agent-containing hydrogel in terms of the area of the outgrown cardiac progenitor cells (*, p<0.05 compared to ‘Static’);

FIG. 12 shows immunofluorescence microphotographs of the cells which have grown from human myocardial fragments to hydrogel, expressing cardiomyocyte-specific transcription factors;

FIG. 13 shows microphotographs of cells that have grown from human myocardial fragments to an antifibrinolytic agent-containing hydrogel after immunochemical staining for immunological traits;

FIG. 14 shows cardiac progenitor cells grown out of myocardial fragments after organ culture in a three-dimensional pattern within hydrogel containing an antifibrinolytic agent (A) or recovered cardiac progenitor cells from hydrogel after treatment of fibrinolytic agents (B) or cardiac progenitor cells recovered from hydrogel cultured in a two-dimensional pattern (C), and cell yields (D) (*, p<0.01 compared to 2D; #, p<0.01 compared to 3D w/o AMBA);

FIG. 15 shows the colony forming unit (A), the morphology (B), and the population doubling time (C) of cardiac progenitor cells after the cells grown in an antifibrinolytic agent-containing hydrogel were amplified in a monolayer culture condition;

FIG. 16 is a graph showing immunological traits of the cardiac progenitor cells grown in an antifibrinolytic agent-containing hydrogel, as assayed by flow cytometry, after the cells were amplified in a monolayer culture condition;

FIG. 17 shows immunological traits of the cardiac progenitor cells grown in an antifibrinolytic agent-containing hydrogel in a quantitative manner (A) and in immunofluorescence photographs (B) after the cells were amplified in a monolayer culture condition;

FIG. 18 shows immunological traits of the Nestin-positive cardiac progenitor cells grown in an antifibrinolytic agent-containing hydrogel in a quantitative manner (A) and in immunofluorescence photographs (B) after the cells were amplified in a monolayer culture condition;

FIG. 19 shows immunofluorescence photographs of the cardiac progenitor cells grown in an antifibrinolytic agent-containing hydrogel in which the formation of cardiospheres and the expression of cardiomyocyte-specific proteins by the cells are explained;

FIG. 20 shows graphs explaining the ability of the cardiac progenitor cells grown in an antifibrinolytic agent-containing hydrogel to form cardiospheres and differentiate into cardiomyoctes after the cardiac progenitor cells were cultured in a suspension culture system;

FIG. 21 shows photographs of cardiomyoctes, adipocytes, osteoblasts, and vascular endothelial cells, all differentiated from single clone-derived cardiac progenitor cells that were amplified in a monolayer culture condition after they were isolated from a hydrogel containing an antifibrinolytic agent;

FIG. 22 shows human antibody arrays indicating angiogenesis factors secreted from the cardiac progenitor cells (CPCs) grown in an antifibrinolytic agent-containing hydrogel after they were amplified in a monolayer culture condition, and relevant tables, with muscle-derived stem cells (MDSCs) serving as a control;

FIG. 23 shows graphs in which proteins secreted from cardiac progenitor cells (CPCs) and muscle-derived stem cells (MDSCs) are quantitatively plotted, after the CPCs were grown in an antifibrinolytic agent-containing hydrogel and amplified in a monolayer culture condition;

FIG. 24 shows photographs of hindlimb muscle tissues of mice damaged by hindlimb ischemia before (left panel) and after (right panel) cardiac progenitor cells that had grown in an antifibrinolytic agent-containing hydrogel and amplified in a monolayer culture condition were introduced into the ischemic muscles, in which the cells induced significant regeneration of skeletal muscles;

FIG. 25 shows the revascularization activity of the cardiac progenitor cells that had grown in an antifibrolytic agent-containing hydrogel and been amplified in a monolayer culture condition, in terms of microvessel density, in photographs (B) and in a graph (A) wherein the density of CD34-positive microvessels are remarkably increased in a group injected with the cardiac progenitor cells (w/ CPCs), compared to a non-treated group (w/o CPCs) (*, p<0.05, compared to ‘W/0 CPCs’);

FIG. 26 is a graph in which blood flow rates in mouse models of hindlimb ischemia injected with (w/ CPCs) or without (w/o CPCs) cardiac progenitor cells that had grown in an antifibrinolytic agent-containing hydrogel and been amplified in a monolayer culture condition, with a significant increase in the blood flow of the injected mice, compared to the non-injected mice (*, p<0.05, compared to ‘W/0 CPCs’; **, p<0.01, compared to ‘W/0 CPCs’);

FIG. 27 shows photographs of the differentiation into vascular endothelial cells of the cardiac progenitor cells that had grown in an antifibrinolytic agent-containing hydrogel and been amplified in a monolayer culture condition after the cardiac progenitor cells were injected into an ischemic hindlimb model;

FIG. 28 shows the effect of hydrogel on the delivery of cardiac progenitor cells to the myocardium in an ischemic myocardial infarction model in terms of cell retention ratio between cardiac progenitor cells injected alone or in combination with hydrogel, said cardiac progenitor cells being grown in an antifibrinolytic agent-containing hydrogel and amplified in a monolayer culture condition (*, p<0.05, compared to ‘CPCs’);

FIG. 29 shows the effect of hydrogel on the myocardial regeneration ability of human cardiac progenitor cells (hCPCs) injected to ischemic myocardial infarction models in terms of left ventricle (LV) thickness and fibrotic area, as visualized by collagen staining in the heart excised two weeks after the introduction of myocardial infarction (*, p<0.05, compared to control; #, p<0.05, compared to CPCs);

FIG. 30 shows the effect of hydrogel on the revascularization ability of human cardiac progenitor cells (hCPCs) injected to ischemic myocardial infarction models (*, p<0.05, compared to control; #, p<0.05, compared to CPCs);

FIG. 31 shows immunofluorescence photographs of human cardiac progenitor cells embedded into an antifibrolytic agent-containing hydrogel (hCPCs+H) which were differentiated into cardiomyocytes in the heart after human cardiac progenitor cells (CPCs) were injected into ischemic myocardial infarction models;

FIG. 32 shows immunofluorescence photographs of human cardiac progenitor cells embedded into an antifibrolytic agent-containing hydrogel (hCPCs+H) which were differentiated into vascular smooth muscle cells in the heart after human cardiac progenitor cells (CPCs) were injected into ischemic myocardial infarction models; and

FIG. 33 shows immunofluorescence photographs of human cardiac progenitor cells embedded into an antifibrolytic agent-containing hydrogel (hCPCs+H) which were differentiated into vascular endothelial cells in the heart after human cardiac progenitor cells (CPCs) were injected into ischemic myocardial infarction models.

### DESCRIPTION OF THE PREFERRED EMBODIMENTS

The present invention pertains to myocardium-resident cardiac progenitor cells, a method for culturing the same, a method for the differentiation thereof, a cell therapeutic agent comprising the same, and a therapeutic agent for heart diseases.

Below, a detailed description will be given of the present invention.

Hematopoietic stem cells or mesenchymal stem cells are used as therapeutics for intractable myocardial diseases. Hematopoietic stem cells are difficult to amplify in vitro. As much as 10 L of bone marrow is required to acquire an effective volume of hematopoietic stem cells. When a solid organ is used as a source, the acquisition of mesenchymal stem cells requires tissue dissociation and secondary purification processes. Although obtained after these processes, mesenchymal cells are found at a frequency of one per one million nucleated cells in bone marrow or adipose tissues. Less than 1% of the bone marrow- or adipose-derived mesenchymal stem cells are known to form colonies. As mentioned, there is a limitation in amplifying hematopoietic stem cells and mesenchymal stem cells in vitro to the extent necessary for use in cell therapeutics.

Leading to the present invention, intensive and thorough research into cell therapy for intractable myocardial diseases, aiming to overcome the problems encountered in the prior art, cumulated in the finding that hydrogel allows cardiac progenitor cells, capable of growing effectively in vitro, to be isolated at a high yield, without heart muscle tissue dissociation and secondary purification.

In accordance with an aspect thereof, the present invention addresses a method for culturing myocardium-resident cardiac progenitor cells, comprising: embedding myocardial fragments into hydrogel; culturing the myocardial fragment embedded into hydrogel; degrading only the hydrogel to recover cardiac progenitor cells grown within the hydrogel; and amplifying the cardiac progenitor cells in vitro.

In one embodiment of the culturing method of the present invention, 1) a myocardial fragment is embedded into fibrin hydrogel containing an antifibrinolytic agent selected from the group consisting of aminocaproid acid, tranexamic acid, and a combination thereof; 2) the myocardial fragment embedded into fibrin hydrogel is three-dimensionally organ cultured while shaking at 5 to 30 rpm to allow muscle-resident cardiac progenitor cells to grow out of the myocardial fragment in the hydrogel; 3) the fibrin hydrogel is degraded by an enzyme selected from the group consisting of urokinase, streptokinase, plasmin, and a combination thereof to recover the myocardium-resident cardiac progenitor cells, and the myocardial fragment; and 4) the cardiac progenitor cells recovered from the hydrogel are amplified in a monolayer culture manner.

As used herein, the term “culturing method” is intended to encompass both the separation and the culture of cardiac progenitor cells.

No particular limitations are imparted with regard to the myocardial fragment used in the present invention. Preferably used is a myocardial fragment obtained by sectioning the cardiac muscles to a certain size from which anatomic architectures inhibitory of the migration of cardiac progenitor cells, such as an endocardium and an epicardium, have been removed. Since tissue-resident stem cells are found predominantly in the walls of microvessels, endocardia and epicardia that act as a blockage against the migration of cardiac progenitor cells are preferably removed to allow the direct contact of muscular microvessels with hydrogel, thereby obtaining cardiac progenitor cells at a high yield.

Hydrogel is a three-dimensional net structure in which hydrophilic polymers are cross-linked with each other through covalent or non-covalent bonds. It is capable of phase transition. In a liquid state, hydrogel is homogeneously mixed with myocardial fragments, after which the hydrogel may undergo phase transition into a solid to provide a stable, three-dimensional, physical support for the myocardial fragments. In addition, the hydrogel support serves as a three-dimensional matrix in which myocardium-resident cardiac progenitor cells grow out of the myocardial fragments.

After being mixed with the myocardial fragments, a hydrosol, a hydrogel in a liquid state, is polymerized and cross-linked to form a hydrogel. The rate of phase transition from hydrosol to hydrogel may be controlled by adjusting concentrations of a polymerizing agent and a cross-linker, as well as reaction temperatures.

For use as a three-dimensional cell adhesion matrix, the hydrogel may contain an integrin-β1-binding receptor which helps cardiac progenitor cells continuously grow in the hydrogel.

FAK is phosphorylated in response to the engagement of cells with extracellular matrix and integrin-β1, activating the cell signaling pathway which leads to cell division. In this context, a larger area in which cells from the myocardial fragment adhere to an extracellular matrix induces higher cell signaling, resulting in an increase in cell division. The hydrogel provides a three-dimensional cell adhesion matrix that is larger than the limited two-dimensional cell adhesion matrix typically used in monolayer culture, guaranteeing sufficient space where cardiac progenitor cells grow. In the hydrogel, thus, cells can be cultured for a long period of time without intercellular contact inhibition.

No particular limitations are imparted with regard to kinds of the hydrogel. The hydrogel may be made of a polymer selected from the group consisting of a natural polymer, a synthetic polymer, a copolymer of various polymers, and a combination thereof. Preferable is a natural polymer.

Examples of the polymer for use as a material of the hydrogel include collagen, gelatin, chondroitin, hyaluronic acid, alginic acid, Matrigel™, chitosan, a peptide, fibrin, PGA (polyglycolic acid), PLA (polylactic acid), PEG (polyethylene glycol), polyacrylamide, and a combination thereof, with preference for a natural polymer selected from the group consisting of collagen, fibrin, Matrigel, gelatin, and a combination thereof.

When consisting of a synthetic polymer or copolymer, hydrogel exhibits high physical performance thanks to its endurance against degradation for a long period of time, but is poor in biological function as it is somewhat resistant to the migration and growth of cells. On the other hand, the hydrogel consisting of a natural polymer such as collagen or fibrin is highly biocompatible so that it provides an suitable environment for the locomotion and growth of cells while being physically more vulnerable to fibrinolysins secreted from organs or cells, such as tPA (tissue plasminogen activator) and uPA (urokinase plasminogen activator) than that consisting of a synthetic polymer or copolymer.

In the present invention, the hydrogel is made of a natural polymer which is biocompatible to guarantee the locomotion and growth of cells, and contains an antifibrinolytic agent to overcome the physical vulnerability to fibrinolytic degradation.

Functioning to inhibit the fibrinolysis of such a fibrolysin secreted from cells or tissues as tPA or uPA, an antifibrinolytic agent, when contained in hydrogel, makes the hydrogel resistant to the fibrinolytic degradation by tPA or uPA for a long period of time, during which a sufficient number of cardiac progenitor cells can grow out of the myocardial fragments to the hydrogel. Thus, in the presence of an antifibrinolytic agent, the hydrogel can serve as a cell adhesion matrix that is absolutely necessary for the migration and growth of cells during the organ culture of the myocardial fragments.

The heart exhibits high fibrinolytic activity of tPA or uPA compared to other organs such as the stomach, the intestine, bone marrow, and adipose tissues. A hydrogel without an antifibrinolytic agent, as will be illustrated in the following Example section, was degraded by the uPA/tPA released from the myocardial fragment to form a halo, without the observations of cells growing in the hydrogel while the cells adhered only to the surface of the culture vessel, and were grown. In contrast, the hydrogel containing an antifibrinolytic agent was observed to keep the function as a three-dimensional substrate in which the locomotion and growth of cardiac progenitor cells took place. The number of cardiac progenitor cells harvested after the three-dimensional organ culture of 1 mg of a myocardial fragment for 7 days on an antifibrinolytic-containing hydrogel support (3D w/ AMBA) was 1.7×10⁷ cells, which was 10-fold more abundant than that obtained upon three-dimensional organ culture on an antifibrinolytic-void hydrogel (3D w/o AMBA).

Examples of the antifibrinolytic agent useful in the present invention include, but are not limited to, aminocaproid acid, tranexamic acid, aprotinin, aminomethylbenzoic acid, and a combination thereof, with preference for tranexamic acid and/or aminomethylbenzoic acid.

Being three times as high in antifibrinolytic activity as aminocaproid acid or aprotinin, aminomethylbenzoic acid or tranexamic acid may be more suitable for aiding the physical function of the fibrin hydrogel.

The concentration of the antifibrinolytic agent in hydrogel is not particularly limited. The antifibrinolytic agent may range in concentration per 1 ml of hydrogel from 10 to 1000 μg, preferably from 30 to 450 μg, and most preferably from 50 to 200 μg.

At smaller concentrations, the antifibrinolytic agent is less toxic to cardiac progenitor cells, whereas a higher concentration of the antifibrinolytic agent more effectively inhibits the activity of fibrolysins released from cardiac muscles, allowing the hydrogel to serve as an intact three-dimensional cell adhesion matrix for a longer period of time. Therefore, both the cytotoxicity and the antifibrinolytic activity must be taken into consideration in determining a suitable concentration of the antifibrinolytic agent. When too small an amount of an antifibrinolytic agent is used, the fibrinolytic activity is not sufficiently suppressed, so that the hydrogel is degraded, leading to the inhibition of the locomotion and growth of cardiac progenitor cells. On the other hand, too high an amount of the antifibrinolytic agent suppresses fibrinolytic activity, but exerts cytotoxicity on cardiac muscles and cardiac progenitor cells.

In the fibrin hydrogel containing an antifibrinolytic agent, fibrinogen may preferably be present at a concentration of from 0.8 to 5.0 mg/ml, and more preferably at a concentration of from 1.0 to 3.5 mg/ml.

For example, the fibrin may be prepared using fibrinogen at a concentration of from 1.25 to 2.5 mg/ml in the presence of thrombin at a concentration of from 0.1 to 2.5 units/ml.

When too high a concentration of fibrinogen is used, the resulting fibrin hydrogel has a dense microarchitecture with a small pore volume formed therein, and becomes highly resistant to degradation, but causes a significant decrease in the growth of cells. In contrast, a fibrin hydrogel with too small a concentration of fibrinogen is vulnerable to degradation by tPA or uPA, secreted from the cells or myocardial fragments, during the organ culture, and thus is apt to lose the function of the three-dimensional cell adhesion matrix essential for the migration and growth of cells.

As used herein, the term “culture medium” refers to a medium capable of inducing the mobilization, and growth of cardiac progenitor cells ex vivo, and is intended to encompass all media typically used in the culture of mammal cells. The culture medium useful in the present invention may be commercially available as exemplified by Dulbecco's Minimum Essential Medium (DMEM), RPMI, Hams F-10, Hams F-12, α-Minimal Essential Medium (α-MEM), Glasgow's Minimal Essential Medium, and Iscove's Modified Dulbecco's Medium.

In one embodiment of the present invention, the culture medium may comprise a growth factor promotive of the mobilization and growth of cardiac progenitor cells. Examples of the growth factor may include serum, e.g., serum from animals including humans, basic fibroblastic growth factor (bFGF), vascular endothelial growth factor (VEGF), insulin, epidermal growth factor (EGF), leukemia inhibitory factor (LIF), insulin-like growth factor (IGF), platelet-derived growth factor (PDGF), and stem cell factor (SCF). In addition, the culture medium may contain an antibiotic such as penicillin, streptomycin, gentamycin, etc.

According to one embodiment of the present invention, the culture medium may be a DMEM:Hams F12 (1:1) medium supplemented with fetal bovine serum, EGF, bFGF, IGF, and gentamycin. Particularly, the cell culture may comprise the DMEM:Hams F12 (1:1) medium in an amount of from 70 to 95 vol/vol %, fetal bovine serum in an amount of from 5 to 15 vol/vol %, EGF in an amount of from 5 to 50 ng/ml, bFGF in an amount of from 0.5 to 10 ng/ml, IGF in an amount of from 5 to 50 ng/ml, and gentamycin in an amount of from 5 to 50 ng/ml.

To promote the supply of oxygen and nutrients, the three-dimensional culture of organ fragments is conventionally carried out in an air exposure manner or in a dynamic manner on an orbital shaker. The air exposure method is undesirable for long-term culture since the supply of nutrients is limited. On the other hand, the dynamic method causes the organ fragments to undergo shearing damage due to the vortex of the medium.

However, myocardial fragments embedded into the hydrogel of the present invention can be cultured on an orbital shaker to promote the growth of cardiac progenitor cells not only because the hydrogel protects the myocardial fragments from shearing, but also because oxygen and nutrients are sufficiently supplied thereto.

The shaking speed is not particularly limited, but is preferably set to be between 5 and 30 rpm.

For example, when the shaking speed is too low, sufficient supply of oxygen and nutrients cannot be achieved. On the other hand, too high a shaking speed may cause shearing damage to the myocardial fragment, impairing the safety of the myocardial fragments within the hydrogel.

Selective degradation of the hydrogel can be accomplished using a degradation enzyme specific to the component polymers of the hydrogel. Examples of the polymer-specific degradation enzyme include, but are not limited to, collagenase, gelatinase, urokinase, streptokinase, TPA (tissue plasminogen activator), plasmin, hyaluronidase, and a combination thereof.

In one embodiment of the present invention, when myocardial fragments are subjected to three-dimensional organ culture in a collagen hydrogel, a gelatin hydrogel or a Matrigel hydrogel, the hydrogel may be selectively degraded by adding collagenase or gelatinase to the medium. A fibrin hydrogel can be selectively degraded with an enzyme selected from the group consisting of urokinase, streptokinase, plasmin, or a combination thereof. Hyaluronidase may be employed to selectively degrade hyaluronic acid hydrogel.

In the presence of the degradation enzymes specific for hydrogel polymers, neither the cardiac progenitor cells nor the myocardial fragments are structurally destroyed and exposed to cytotoxicity. More than 95% of the recovered cells were observed to survive.

For use in the selective degradation of collagen or gelatin hydrogel, for example, collagenase may be used in an amount of from 0.1 to 1 mg per 1 ml of the collagen or gelatin hydrogel.

Urokinase, streptokinase, or plasmin does not destroy structural components of cardiac progenitor cells or myocardial fragments, but selectively degrades fibrin. Urokinase or streptokinase may be added in an amount of from 100 to 10,000 units per ml of hydrogel.

According to one embodiment of the present invention, incubation at 30 to 37° C. for 0.5 to 3 hrs is needed to promote the enzymatic effect of urokinase or streptokinase.

Following the selective enzymatic degradation of hydrogel, the cardiac progenitor cells which have grown out of the mycocardium fragments in the hydrogel, and the myocardial fragments that have remained structurally intact, can be recovered.

The cardiac progenitor cells recovered from the hydrogel can be amplified. The amplification may be accomplished by, but is not limited to, a monolayer culture method. For instance, the cardiac progenitor cells recovered from the hydrogel are seeded into a culture vessel and grown to 60 to 90% confluence. Then, they are detached by treatment with trypsin-EDTA, and subcultured in a new culture vessel. This passage procedure is repeated to amplify the cells to the number necessary for a therapeutic dose.

In addition, the myocardial fragments recovered by selectively degrading the hydrogel can be embedded into a fresh hydrogel and subjected again to a three-dimensional organ culture to separate and grow cardiac progenitor cells. Like this, the myocardial fragments can be recovered intact.

The recovery of myocardial fragments that remain structurally intact is of significance. A cardiac muscle sample can be taken using cardiac catheterization, or a biopsy method, but only in a small quantity. Hence, it is an important factor to stably and effectively isolate and grow cardiac progenitor cells from a small quantity of cardiac tissues.

As described above, once taken from a patient with a heart disease, a cardiac muscle sample, even in a small quantity, can be repeatedly used many times in the culturing method using hydrogel in accordance with the present invention, so that it allows for the production of cardiac progenitor cells to the number necessary for a therapeutic dose for the heart disease without the need to repeatedly take cardiac muscle samples.

In the present invention, the time of the three-dimensional organ culture is not particularly limited, but preferably ranges from 1 to 28 days, and more preferably from 3 to 14 days. The three-dimensional organ culture of a myocardial fragment on the hydrogel support may induce cardiac progenitor cells to grow out of the myocardial fragment in the hydrogel from 12 hours after culturing.

In accordance with a further aspect thereof, the present invention addresses cardiac progenitor cells, obtained using the culturing method of myocardium-resident cardiac progenitor cells of the present invention, which exhibit at least one immunological trait of (i) being positive to a cardiac progenitor cell marker selected from the group consisting of nestin, Sca-1 and a combination thereof; (ii) being positive to a cardiomyocyte-specific transcription factor marker selected from the group consisting of GATA-4, Nkx-2.5, Mef-2c, and a combination thereof; (iii) being positive to a mesenchymal stem cell marker selected from the group consisting of CD29, CD44, CD73, CD90, CD105, and a combination thereof; (iv) being positive to a vascular pericyte marker selected from the group consisting of CD140b, CD146, α-smooth muscle actin (SMA), and a combination thereof; (v) being negative to a hematopoietic cell marker selected from Lin, CD34, CD45, and a combination thereof; (vi) being negative to a vascular endothelial cell marker selected from the group consisting of CD31, CD34, and a combination thereof; and (vii) being negative to a cardiomyocyte marker selected from the group consisting of α-SA (α-sarcometric actinin), MHC (myosin heavy chain), TnI (troponin I), TnT (troponin T), and a combination thereof.

Cardiomyocytes account for 20%˜30% of cardiac cells while the remaining 70%˜80% is comprised of fibroblasts, smooth muscle cells, vascular endothelial cells, hematopoietic cells, and cardiac progenitor cells. Of the heart cells, only about 0.03% of cells are accounted for by cardiac progenitor cells. Thus, a typical method comprises treating a heart muscle sample with a degradation enzyme, such as collagenase, to separate single cardiac cells, and immunological purification cardiac progenitor cells from heterogeneous cell groups. The tissue dissociation is difficult to standardize. The quantity of single cells during the tissue dissociation process is greatly influenced by the kind of enzymes used, titer, reaction time, reaction temperature, and state of tissues used. Moreover, the cells cannot avoid being damaged during the tissue dissociation process. Of the single cells obtained using a conventional method, only 0.03%˜0.7% are cardiac progenitor cells (0.03˜0.08%, disclosed in [paragraph-0168] of WO 2004/019767; 0.7%, disclosed in Circ Res 2011; 108: 857). Thus, a subsequent immunological process is needed to isolate cardiac progenitor cells from the other cells. In contrast, the culturing method of the present invention allows the isolation of cardiac progenitor cells in a high purity without an additional immunological purification, and does not cause cellular damage because it involves no tissue dissociation processes. More than 95% of the recovered cells were observed to survive. After organ culture of 1 gm of a myocardial fragment for 7 days, 1.7×10⁷ cardiac progenitor cells were obtained using the method of the present invention. Therefore, the method of the present invention is overwhelmingly advantageous over conventional methods in terms of yield.

The cardiac progenitor cells obtained using the culturing method of myocardium-resident cardiac progenitor cells in accordance with the present invention exhibit at least one immunological trait of (i) being positive to a cardiac progenitor cell marker selected from the group consisting of nestin, Sca-1, and a combination thereof; (ii) being positive to a cardiomyocyte-specific transcription factor marker selected from the group consisting of GATA-4, Nkx-2.5, Mef-2c, and a combination thereof; (iii) being positive to a mesenchymal stem cell marker selected from the group consisting of CD29, CD44, CD73, CD90, CD105, and a combination thereof; (iv) being positive to a vascular pericyte marker selected from the group consisting of CD140b, CD146, α-smooth muscle actin (SMA), and a combination thereof; (v) being negative to a hematopoietic cell marker selected from Lin, CD34, CD45, and a combination thereof; (vi) being negative to a vascular endothelial cell marker selected from the group consisting of CD31, CD34, and a combination thereof; or (vii) being negative to a cardiomyocyte marker selected from the group consisting of α-SA (α-sarcometric actinin), MHC (myosin heavy chain), TnI (troponin I), TnT (troponin T), and a combination thereof.

Of the cells grown in the hydrogel when using the culturing method of cardiac progenitor cells according to the present invention, more than 95% of cells are observed to express a cardiac progenitor cell marker such as nestin or Sca-1 while being positive to the cardiomyocyte-specific transcription factor GATA-4, Nkx-2.5, or MEF-2c. In contrast, the cells may comprise somatic cells such as vascular endothelial cells, hematopoietic cells, smooth muscle cells, etc. in an amount of less than 1%, and may express none of the cardiomyocyte markers α-SA, TNI, desmin, and MHC.

In addition, the cardiac progenitor cells express at least one mesenchymal stem cell marker selected from the group consisting of CD29, CD44, CD73, CD90, and CD105, and at least one vascular pericyte marker selected from the group consisting of CD140b, CD146, and SMA while being negative to all of the hematopoietic cell markers Lin, CD34, CD45, and CD56. In addition, the cardiac progenitor cells obtained by the method of the present invention express neither CD 56, a marker for both neural cells and mature cardiomyocytes, nor CD31 and CD34, markers for vascular endothelial cells.

The cardiac progenitor cells of the present invention exhibit the same immunological traits as those of vascular pericyte-derived mesenchymal stem cells. In addition, the cardiac progenitor cells are differentiated ex vivo in a similar pattern as in the bone marrow-derived mesenchymal stem cells. Hence, the myocardium-resident cardiac progenitor cells may be classified as cardiac muscle-derived mesenchymal stem cells.

The cardiac progenitor cells may have a high potential to differentiate into various tissues and organs. For example, they can differentiate into cariomyocytes, osteoblasts, adipocytes, chondrocytes, vascular endothelial cells, smooth muscle cells, neural cells, or skeletal muscle cells.

Differentiation of the cardiac progenitor cells into cells selected from the group consisting of skeletal muscle cells, osteoblasts, adipocytes, chondrocytes, vascular endothelial cells, neural cells, and a combination thereof can be implemented in a differentiation-inducing condition or medium known in the art.

Cardiac progenitor cells are capable of directly differentiating into cardiomyocytes, whereas the direct differentiation of hematopoietic stem cells and mesenchymal stem cells to cardiomyocytes has not yet been proven.

Further, the cardiac progenitor cells isolated using conventional methods are capable of spontaneously differentiating into cardiomyocytes only under limited conditions. Less than 10% of the total cardiac progenitor cells are known to differentiate into cardiomyocytes, whereas the myocardium-resident cardiac progenitor cells of the present invention can spontaneously differentiate into cardiomyocytes in more than 70% of the total population, into vascular endothelial cells in more than 85% of the total population, and into smooth muscle cells in 20% of the total population.

The cardiac progenitor cells isolated and cultured by the method of the present invention exhibit excellent ex vivo growth performance. For example, about 70% of them can form colonies, and undergo 200 or more rounds of cell division, with a doubling time of 30 to 60 hrs.

In accordance with still another aspect thereof, the present invention addresses a method for differentiating myocardium-resident cardiac progenitor cells into cardiomyocytes, comprising culturing the cardiac progenitor cells in a suspension cell culture process.

Exhibiting the same differentiation properties as bone marrow- or adipose-derived mesenchymal stem cells, the cardiac progenitor cells of the present invention not only differentiate into particularly limited cells, but can differentiate into at least one cell selected from the group consisting of an osteoblast, an adipocyte, a chondrocyte, a vascular endothelial cell, a smooth muscle cell, a neural cell, and a skeletal muscle cell.

No particular limitations are imparted with regard to the cells into which the cardiac progenitor cells spontaneously differentiate. Preferably, the cardiac progenitor cells spontaneously differentiate into cardiomyocytes.

The spontaneous differentiation of the cardiac progenitor cells isolated using a conventional method into cariomyocytes is limited. Less than 10% of the total cardiac progenitor cells are known to spontaneously differentiate into cardiomyocytes. In contrast, the cardiac progenitor cells of the present invention can differentiate into cardiomyocytes in a spontaneous manner in more than 70% of the total population, into vascular endothelial cells in more than 85% of the total population, and into smooth muscle cells in 20% of the total population. Mesenchymal stem cells and hematopoietic stem cells, conventionally used as cell therapeutics for the regeneration of cardiac muscles, indirectly protect the heart, but cannot directly regenerate cardiac muscles because they lack the potential to differentiate into cardiac cells. In contrast, the cardiac progenitor cells of the present invention have the potential to directly and spontaneously differentiate into all cardiac cells, exhibiting applicability to the use as a source of cell therapeutics for cardiac regeneration.

The differentiation of cardiac progenitor cells into cardiomyocytes may be induced by co-culturing with cardiomyocytes in a monolayer culture condition or by culturing in the presence of 5-azacytidine or oxytocin. These conventional methods are, however, limited in the performance of inducing differentiation into cardiomyocytes, and are also ineffective in assaying the potential of a certain stem cell into cardiomyocytes. In the body, all organs and cells exist in three-dimensional structures. Cells perform their intrinsic biological functions through interaction between cells and between cells and extracellular matrixes. A conventional monolayer culture environment does not mimic the in vivo environment, and cannot guarantee interactions between cells or between cells and extracellular matrixes, which results in a failure to effectively and stably induce differentiation into cardiomyocytes.

According to a suspension cell culture process, cardiac progenitor cells are suspended in a culture medium and aligned in a three-dimensional structure in a culture vessel designed to prevent cell adhesion thereto, so that the cells can take three-dimensional microarchitectures through cell-to-cell and cell-to-extracellular matrix interactions. In addition, the suspension cell culture process can induce the spontaneous differentiation of the cardiac progenitor cells into cardiomyocytes without the aid of a particular exogenous inducing agent.

For instance, when 1000 cardiac progenitor cells are suspended in 1 ml of a culture medium in a culture vessel which is previously treated to prevent cell adhesion thereto, they are aligned in a three-dimensional structure. Following construction of a three-dimensional alignment thereof, the cells are cultured for one day to four weeks, and preferably for three days to two weeks, to induce differentiation into cardiomyocytes. Thus, the suspension cell culture can support interactions between cells and between cells and extracellular matrixes through a three-dimensional architecture.

Further, the suspension cell culture process may be utilized as a system for assaying the potential of cardiac progenitor cells, differentiable somatic cells, embryonic stem cells, or induced pluripotent stem cells to differentiate into cardiomyocytes, and for assaying the ability of an agent or factor to induce differentiation into cardiomyocytes.

The cardiomyocytes differentiated in the suspension cell culture process may exhibit the immunological trait of being positive to a marker selected from the group consisting of α-SA, TnI (troponin I), TnT (troponin T), α-MHC (α-myosin heavy chain), β-MHC (β-myosin heavy chain), MLC2a (myosin light chain-2 atrium), MLC2v (myosin light chain-2 ventricle), and a combination thereof.

In accordance with a still further aspect thereof, the present invention addresses a cell therapeutic agent, comprising the cardiac progenitor cells or the cells differentiated therefrom as an active ingredient.

Cardiac progenitor cells may be utilized as a cell therapeutic agent as they are, without a special differentiation procedure, or may be differentiated into target cells for use as a cell therapeutic agent.

No particular limitations are imparted with regard to the cells of the cell therapeutic agent. Examples of the cells which treated by the cell therapeutic agent of the present invention include, but are not limited to, cardiomyocytes, osteoblasts, adipocytes, chondrocyte, vascular endothelial cells, smooth muscle cells, neural cells, and skeletal muscle cells.

Non-limiting, typical methods may be used to apply the cardiac progenitor cells or their differentiated cells as a cell therapeutic agent. Preferably, the cardiac progenitor cells may be administered in conjunction with a biodegradable support or carrier.

The biodegradable support or carrier causes no substantial toxicity in the host, and can be biologically degraded. It can be naturally removed from and/or chemically incorporated into a biological system.

The biodegradable support or carrier is not particularly limited with regard to its kind, and may preferably be a hydrogel, and more preferably a hydrogel containing an antifibrinolytic agent.

The cardiac progenitor cells may be used in a mixture with a hydrogel containing an antifibrinolytic agent.

Concentrations and kinds of the constituent polymers and the antifibrinolytic are factors determining the biodegradation rate of the hydrogel. In a mixture with the hydrogel, the cells are prevented from being lost by the blood stream and protected from being damaged at the lesion by inflammatory cells and enzymes.

In an alternative embodiment, the cell therapeutic agent may further comprise a factor selected from the group consisting of an anti-inflammatory agent, a stem cell mobilizing factor, a vascular growth factor, and a combination thereof.

The anti-inflammatory agent is adapted to allow the hydrogel to protect the transplanted cardiac progenitor cells from excessive inflammation. The stem cell mobilizing factor or the vascular growth factor can contribute to cell regeneration as well.

Examples of the anti-inflammatory agent may include, but are not limited to, a COX inhibitor, an ACE inhibitor, salicylate, and dexamethasone.

The stem cell mobilizing factor is not particularly limited, and may be selected from the group consisting of IGF, bFGF, PDGF, EGF, and a combination thereof.

No particular limitations are imparted with regard to the vascular growth factor. Examples of the vascular growth factor may include EGF, PDGF, VEGF, ECGF, and angiogenin.

In one embodiment of the present invention, the cell therapeutic agent may further be one or more diluents. Examples of the diluents include, but are not limited to, physiological saline, a buffer such as PBS (phosphate buffered saline) or HBSS (Hank's balanced salt solution), plasma, and a blood ingredient. In addition to the diluent, the cell therapeutic may comprise a lubricant, a wetting agent, a sweetener, a favoring agent, an emulsifier, a suspending agent, and a preservative.

In accordance with yet another aspect thereof, the present invention addresses a pharmaceutical composition for the prophylaxis or therapy of a heart disease, comprising the cardiac progenitor cells or their differentiated cells as an active ingredient.

As a therapeutic dose effective for the therapy of a heart disease, for example, myocardial infarction, an ischemic myocardial disease, a secondary myocardial disease, or a congestive heart failure, the cell therapeutic requires 1×10⁷ to 1×10⁸ cells.

Currently, typical therapeutics for intractable myocardial diseases are hematopoietic stem cells or mesenchymal stem cells. In vitro amplification of hematopoietic stem cells is difficult. As much as 10 L of bone marrow is required to acquire an effective volume of hematopoietic stem cells. Mesenchymal cells are found at a frequency of one per one million nucleated cells in bone marrow or adipose tissues. Less than 1% of the bone marrow- or adipose-derived mesenchymal stem cells are known to form colonies, with a doubling time of 60 hrs or longer. As mentioned, there is a limitation in amplifying hematopoietic stem cells and mesenchymal stem cells in vitro to the extent necessary for use in cell therapeutics. In addition, hematopoietic stem cells and mesenchymal stem cells have not yet been proven to directly differentiate into cardiomyocytes.

Using the culturing method of the present invention, one million cardiac progenitor cells can be isolated within 7 days from 100 mg of a heart muscle. The cardiac progenitor cells can be amplified to ten million cells by performing a monolayer culture for a couple of weeks. In addition, more than 70% of the cultured cardiac progenitor cells can form colonies and undergo as many as 200 rounds of cell division, to produce a therapeutic dose necessary for the therapy of a heart disease within a short time.

In the pharmaceutical composition, the cardiac progenitor cells or their differentiated cells may be used in mixture with a hydrogel containing an antifibrinolytic agent.

Conventionally, therapeutics or stem cells are transplanted directly into a heart muscle lesion or infused into the vessels. Upon direct injection of cell therapeutics into the cardiac muscle, less than 1% of the cells injected were observed to survive in the cardiac muscles while the other cells were damaged by excessive inflammatory cells, enzymes, and hypoxia, or lost by hemorrhage upon injection. Almost all of the cells infused through blood vessels failed to pass through the capillary vessel-abundant organs liver, spleen, and lungs, with only 0.1% of the infused cells arriving at the heart.

When a hydrosol containing an antifibrinolytic agent is mixed with the cardiac progenitor cells and injected into a cardiac muscle lesion, it undergoes a phase transition into a gel which effectively delivers the cardiac progenitor cells to the cardiac muscles. The cardiac progenitor cells are delivered at 5-fold higher efficiency in combination with the hydrogel than alone. Upon transplantation, more than 5% of the cells embedded into a hydrogel containing an antifibrinolytic were found to survive in the cardiac muscle lesion and to exert a direct effect of cardiac muscle regeneration on the lesion.

As will be illustrated in the following Example section, human cardiac progenitor cells were found to occupy less than 5% of the cardiac muscle area when transplanted alone (CPCs), but to be distributed over a 3-fold larger area when transplanted in combination with an antifibrinolytic-induced hydrogel (CPCs+H).

Possessing robust ex vivo expandability and a potential to spontaneously differentiate into cardiac cells, the myocardium-resident cardiac progenitor cells can be effectively applied to the prophylaxis or therapy of a heart disease.

In one embodiment of the present invention, the cardiac progenitor cells injected in combination with a hydrogel can be differentiated into cardiomyocytes, vascular endothelial cells, vascular smooth muscle cells, thus functioning to directly regenerate cardiac muscles and vessels.

The heart disease is not particularly limited, and may preferably be exemplified by acute and chronic myocardial infarction, ischemic myocardial diseases, primary or secondary myocardial disease, and congestive heart failure.

In addition to the cardiac progenitor cells or their differentiated cells as an active ingredient, the pharmaceutical composition of the present invention may further comprise additives typically used in the art, such as carriers, excipients, and diluents. For formulations of the pharmaceutical composition, reference may be made to a method typical to the art (e.g., Remington's Pharmaceutical Science, latest edition; Mack Publishing Company, Easton Pa.).

The pharmaceutical composition may be administered without limitations. For example, it may be formulated into an injection, or may be directly transplanted into a cardiac lesion by surgery or may be injected intravenously. Once administered, the cardiac progenitor cells move towards diseased cardiac tissues.

The pharmaceutical composition may further comprise at least one selected from the group consisting of an anti-inflammatory agent, a stem cell mobilizing factor, and a vascular growth factor.

A better understanding of the present invention may be obtained through the following examples which are set forth to illustrate, but are not to be construed as limiting, the present invention.

### EXAMPLES

### Example 1

### Construction of Hydrogel Microarchitecture with Various Concentrations of Fibrinogen

In order to examine the effect of fibrinogen on the microarchitecture thereof, fibrin hydrogels were constructed with various concentrations of fibrinogens. In this regard, fibrin was prepared from four concentrations of fibrinogen. Human plasma-derived fibrinogen (GreenCross, Seoul, Korea) was dissolved in DMEM containing 10 mM CaCl₂ to give 2.5, 5.0, 10.0, and 20.0 mg/ml fibrinogen solutions. Alexa Fluor 488-conjugated fibrinogen (1:50 w/w) (Invitrogen, Carlsbad, Calif.) was added to the fibrinogen solutions. Separately, thrombin (Sigma, St. Louis, Mich.) was dissolved in DMEM to form a thrombin solution with a final concentration of 1 unit/ml. Each of the four fibrinogen solution was mixed at a ratio of 1:1 (v/v) with the thrombin solution, and 10 μl of each of the resulting mixtures was placed on a glass slide and incubated at 37° C. for 2 hrs for a cross-linking reaction. Finally, four fibrin hydrogels comprising fibrinogen in a concentration of 1.25, 2.5, 5.0, or 10.0 mg/ml and thrombin in a concentration of 0.5 units/ml were obtained and examined for their microarchitectures under a confocal microscope and a scanning electron microscope.

At higher concentrations of fibrinogen, the hydrogels were observed to have denser microarchitectures while the fibrins became thicker, with smaller pore sizes of the hydrogels (FIG. 1). The pore size of the hydrogels was 25 nm at a fibrinogen concentration of 1.25 mg/ml, and was significantly reduced to 12.3 nm and 7.5 nm at fibrinogen concentrations of 5 mg/ml and 10 mg/ml, respectively (p<0.05).

### Example 2

### Fibrinolysis of Cardiac Progenitor Cells

Four hydrogels with different fibrinogen concentrations were prepared in the same manner as in Example 1. Alexa Fluor 488-conjugated fibrinogen (1:50 w/w) (Invitrogen, Carlsbad, Calif.) was added to each of the four fibrinogen solutions. The cardiac progenitor cells were mixed at a density of 2×10⁵ cells per 100 μl of each of the four fibrinogen solutions with 100 μl of the thrombin solution, followed by a cross-linking reaction for 2 hrs. Following the formation of hydrogel, 300 μl of a cell culture was added to the hydrogel, and incubated for 1 day. A hydrogel void of cardiac progenitor cells was prepared for use as a control. Degradation rates of the fibrin contained in hydrogels were determined by measuring the Alexa Fluor 488-conjugate fibrinogen released to the culture medium by means of a fluorometer.

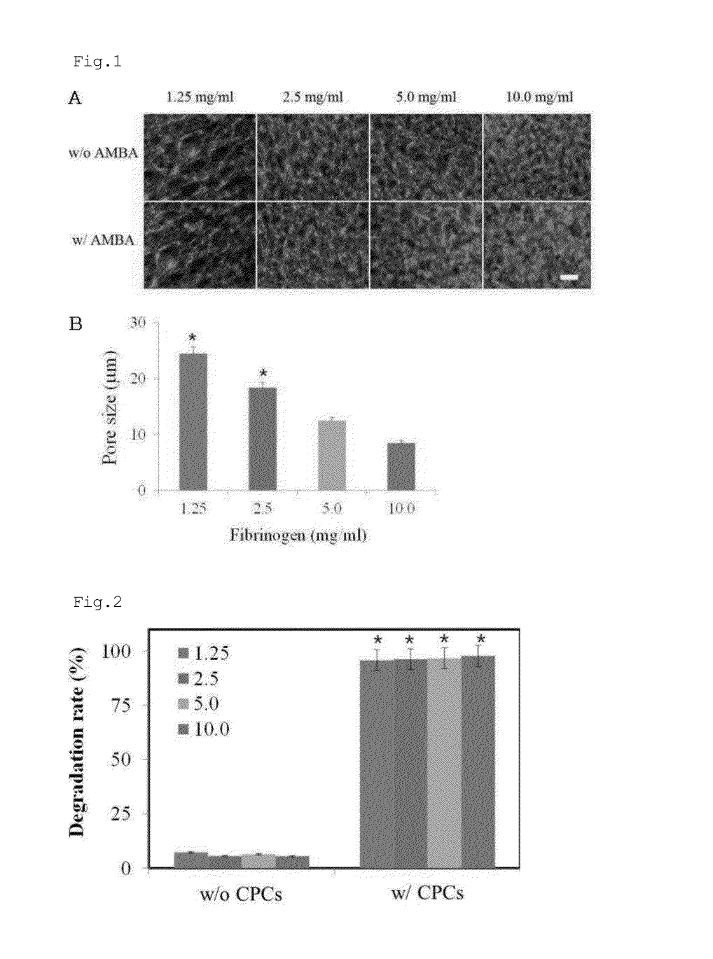

As can be seen in FIG. 2, the hydrogel embedded with cardiac progenitor cells (w/CPCs) started to degrade from 2 hrs after incubation, and completely degraded 1 day after incubation. In contrast, the control was degraded at a rate less than 5%, with no degradation differences observed at different concentrations of fibrinogen.

### Example 3

### Inhibition of Antifibrinolytic Agents against Cardiac Progenitor Cell-Induced Fibrinolysis

Antifibrinolytic agents were purchased from Sigma. Cardiac progenitor cells were embedded in the same manner as in Example 2 into a hydrogel containing an antifibrinolytic agent, and incubated for 1 day before the analysis of fibrin degradation.

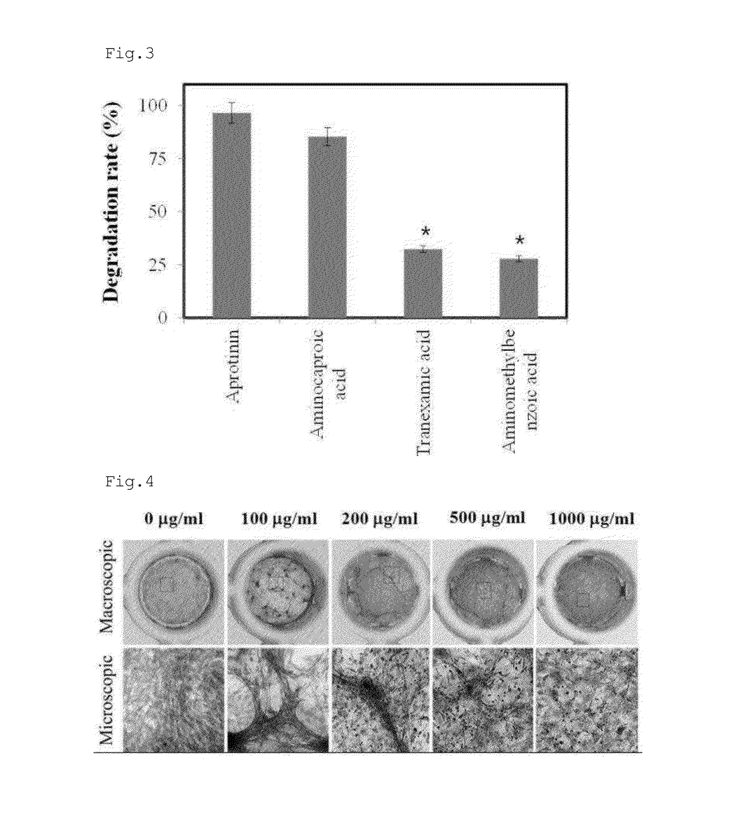

As can be seen in FIG. 3, fibrin hydrogels containing aprotinin or aminocaproic acid were degraded to the degrees of 95% and 80%, respectively, by the cardiac progenitor cells, indicating that aprotinin and aminocaproic acid are slightly inhibitory of cardiac progenitor cell-induced fibrinolysis. In contrast, the hydrogels containing tranexamic acid or aminomethylbenzoic acid were resistant to the fibrinolytic activity of cardiac progenitor cells, as demonstrated by the degradation of the hydrogel at a rate of less than 30%.

### Example 4

### Inhibitory Effect of Concentration of Antifibrinolytic Agent on Cardiac Progenitor Cell-Induced Degradation of Hydrogel

100 μl of the fibrinogen solution containing cardiac progenitor cells was added to 100 μl of each of the respective thrombin solutions containing aminomethylbenzoic acid in concentrations of 0.2, 0.1, 0.2, 0.5, and 1.0 mg/ml. After complete formation of hydrogel, cardiac progenitor cells were cultured for 1 day, fixed with formalin, and stained with toluidine blue. An examination was made of the states of the fibrin hydrogels and cells.

The results are shown in FIG. 4. The hydrogel void of aminomethylbenzoic acid was completely degraded, thus failing to serve as a three-dimensional cell adhesion substrate. In this condition, the cells were grown in a two-dimensional manner on the bottom of the culture vessel. On the other hand, the hydrogels containing aminomethylbenzoic acid in a concentration of 0.1 or 0.2 mg/ml were resistant to the cardiac progenitor cell-induced fibrinolysis, thus serving as a three-dimensional cell adhesion substrate. However, aminomethylbenzoic acid in a concentration of 0.5 or 1.0 mg/ml, although strongly suppressing the cardiac progenitor cell-induced fibrinolysis, caused cytotoxicity to the cardiac progenitor cells.

### Example 5

### Effect of Composition of Hydrogel Containing Antifibrinolytic Agent on Behavior and Growth of Cardiac Progenitor Cell

Four fibrinogen solutions with respective concentrations of 2.5, 5.0, 10.0, and 20.0 mg/ml were prepared in the same manner as in Example 1. Each of the fibrinogen solutions was added with a 100 μg/ml aminomethylbenzoic acid solution, and then mixed at a volume ratio of 1:1 with a thrombin solution containing the cardiac progenitor cells, followed by a cross linking reaction, as described in Example 2. Within the antifibrinolytic agent-containing hydrogel thus formed, the cardiac progenitor cells were cultured for 3 days. The cardiac progenitor cells were examined for cytoplasmic spreading by confocal microscopy and phase-contrast microscopy after reaction with 1 μg/ml Alexa Fluor 488-conjugated Phalloidine (Invitrogen) for 30 min. The growth of the cardiac progenitor cells was evaluated using a dsDNA PicoGreen Quantitation Kit.

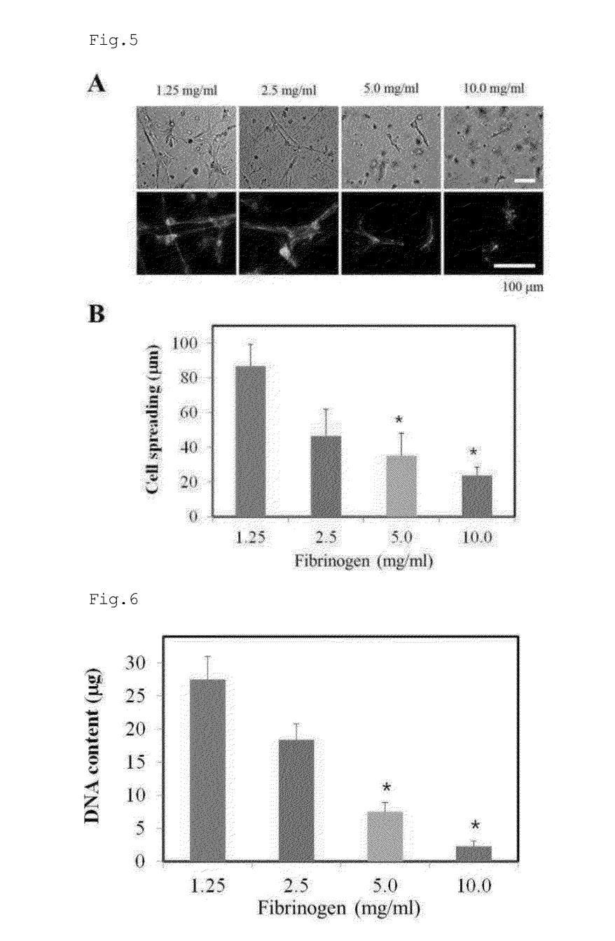

As can be seen in FIG. 5, the cardiac progenitor cells exhibited three-dimensional cytoplasmic spreading in the hydrogel containing fibrinogen in a concentration of 1.25 or 2.5 mg/ml, and an antifibrinolytic agent. On the other hand, the cytoplasmic spreading of the cardiac progenitor cells in the hydrogels containing fibrinogen in a concentration of 5.0 mg/ml or greater, and an antifibrinolytic agent was reduced. Particularly, no cytoplasmic spreading was observed in the hydrogel containing fibrinogen in a concentration of 10.0 mg/ml and an antifibrinolytic agent. Quantitative analysis also showed that the hydrogel containing a physiological concentration of an antifibrinolytic agent allowed the cardiac progenitor cells to perform cytoplasmic spreading to a significantly high degree than did the hydrogel containing fibrinogen in a concentration of cytoplasmic spreading of 5.0 mg/ml or greater (*, p<0.01) (FIG. 5B).

As can be seen in FIG. 6, the growth of the cardiac progenitor cells within the hydrogel containing fibrinogen and an antifibrinolytic agent was significantly decreased with an increase in the concentration of fibrinogen. A significant increase in the growth of the cardiac progenitor cells was observed when the hydrogel contained a physiological concentration of an antifibrolytic agent (*, p<0.01).

### Example 6

### Outgrowth of Myocardial fragment in Antifibrinolytic Agent-Containing Hydrogel of Cardiac Progenitor Cells

Four fibrinogen solutions with respective concentrations of 2.5, 5.0, 10.0, and 20.0 mg/ml were prepared, and each was added with a 100 μg/ml aminomethylbenzoic acid or tranexamic acid solution, as in Example 5. From the heart of a brain-dead patient, the myocardium was taken after the removal of both the epicardium and the endocardium. The cardiac muscle tissue was cut into fragments in a dimension of from 1 to 3 mm³ and washed three times with DMEM. The myocardial fragments were added at a density of 10 per 0.5 ml of a 1 unit/ml thrombin solution. Each of the four fibrinogen solutions was mixed at a volume ratio of 1:1 with the thrombin solution containing the cardiac fragments. The resulting mixture was aliquoted in an amount of 1 ml per well into 6-multiwell tissue culture plates before performing a polymerization and cross-linking reaction to form a hydrogel. To the hydrogel was added 2 ml of a cell culture medium, followed by subjecting the myocardial fragments to organ culture for 3 days. Thereafter, the cultures were fixed with 3% formalin, and cardiac progenitor cells that had grown out of the myocardial fragments in the hydrogel were evaluated using phase-contrast microscopy.

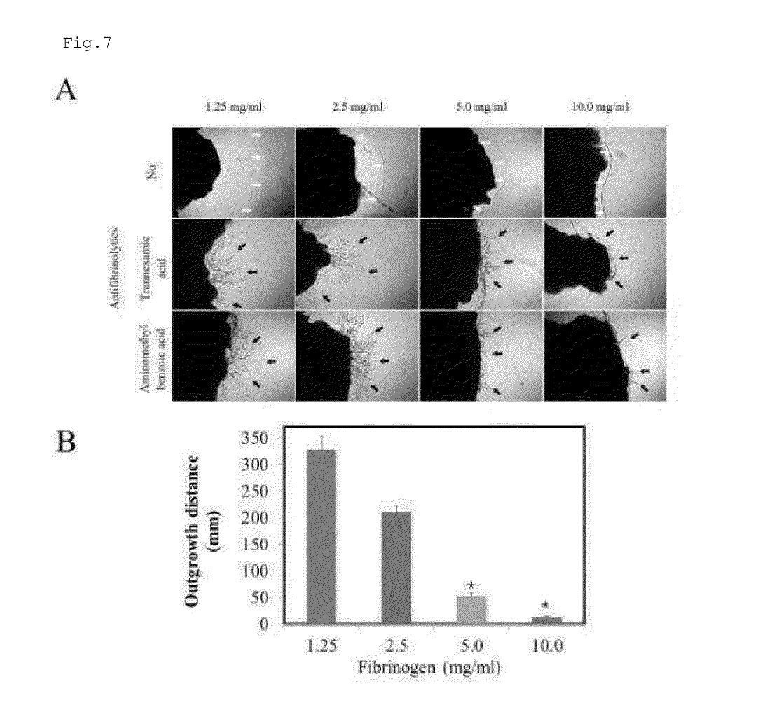

As can be seen in FIG. 7, the hydrogel containing no antifibrinolytic agents was degraded during incubation of the myocardial fragments (upper panels in FIG. 7A). The hydrogels containing low concentrations of fibrinogen were degraded in a larger area than were the hydrogels containing fibrinogen concentrations of 5.0 and 10.0 mg/ml. However, the hydrogels having an antifibrinolytic agent, such as aminomethylbenzoic acid or tranexamic acid, could provide the myocardial fragments with stable, three-dimensional cell adhesion substrates during the organ culture.

Fibrinogen reduced the number of the cardiac progenitor cells that grew out of the myocardial fragments in the hydrogel having an antifibrinolytic agent in a dose-dependent manner (FIG. 7B). The cardiac progenitor cells which grew out of the myocardial fragments in the hydrogel containing an antifibrinolytic agent were observed 1 day before the organ culture when the hydrogel contained fibrinogen was in a concentration of 1.25 mg/ml, but were not observed until 2 days after the organ culture when the hydrogel contained fibrinogen in a concentration of 5 mg/ml. Fibrinogen was found to reduce the outgrowth distance of cardiac progenitor cells that had grown out of the myocardial fragments in the antifibrinolytic agent-containing hydrogel in a dose-dependent manner, as assayed by a morphometric method (p<0.01) (FIG. 7B).

### Example 7

### Three-Dimensional Organ Culture of Myocardial Fragment on Antifibrinolytic-Containing Hydrogel Support

A hydrogel containing 2.0 mg/ml fibrinogen, 0.5 units/ml thrombin, and 100 μg/ml aminomethylbenzoic acid was prepared. Myocardial fragments were subjected to three-dimensional organ culture in the hydrogel. The cell culture medium comprised DMEM in an amount of 90 vol %, fetal bovine serum in an amount of 10 vol %, EGF in an amount of from 20 ng/ml, bFGF in an amount of 5 ng/ml, IGF in an amount of 10 ng/ml, and gentamycin (Invitrogen) in an amount of 10 μg/ml. The organ culture was carried out for one week in a culture vessel on an orbital shaker moving at 15 to 30 rpm, with the culture medium replaced with a fresh medium every two days. The cardiac progenitor cells that had grown out of the myocardial fragments in the hydrogel during the organ culture were recovered, together with the myocardial fragments, by selectively degrading the antifibrolytic-containing hydrogel. The recovered cardiac progenitor cells were amplified by two-dimensional monolayer culture while the recovered myocardial fragments were embedded again into an antifibrinolytic agent-containing hydrogel and subjected to three-dimensional organ culture. The properties of the cells growing out of the myocardial fragment in the antifibrinolytic agent-containing hydrogel, and the structure of the cultured myocardial fragment were examined. In this regard, paraffin blocks were prepared using a conventional method after the organ culture, and subjected to hematoxylin eosin staining and immunohistochemical staining. To trace the cells that grew out of the myocardial fragment in the hydrogel, incubation with 1 μM bromodeoxyuridine (BrdU, Sigma) was carried out for 3 days of the one week of organ culture. The uptake of BrdU was detected by immunochemical staining. Before being cultured, myocardial fragments were either seeded (2D) or embedded into an antifibrinolytic hydrogel (3D). One day after organ culture, only the myocardial fragments were recovered. Proteins were extracted from the myocardial fragments and used in Western blotting analysis for the integrin signaling pathway.

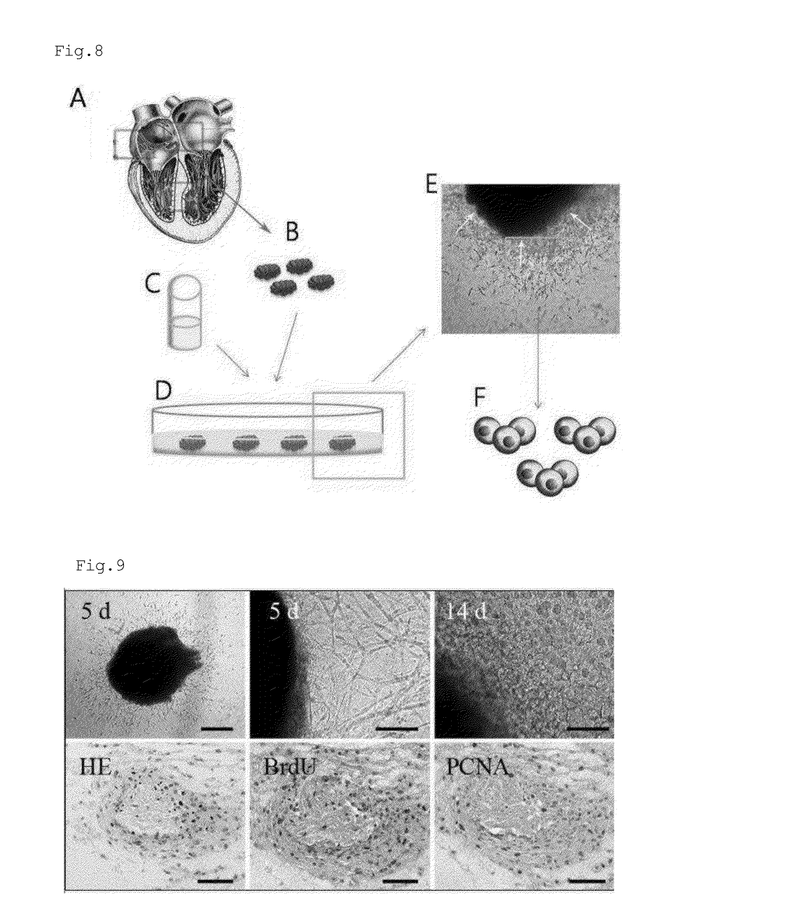

As seen in FIG. 9, cardiac progenitor cells which grew out of the myocardial fragments in the antifibrinolytic agent-containing hydrogel were observed 1 day before the organ culture. The cells that grew in the antifibrinolytic agent-containing hydrogel took a spindle shape like typical fibroblasts (FIG. 8E). After selective degradation of the antifibrinolytic agent-containing hydrogel, the cardiac progenitor cells were stably recovered and seeded into culture vessels. More than 90% of the seeded cells were observed to adhere to the vessels. The cells that grew in the antifibrinolytic hydrogel during 7 days of the organ culture were observed to take a spindle shape (FIG. 9). More than 90% of the cells that grew out of the myocardial fragments in the antifibrinolytic agent-containing hydrogel were observed to express proliferating cell nuclear antigen (PCNA) as well as BrdU, demonstrating in vitro cell division during the organ culture (BrdU and PCNA panels in FIG. 9).

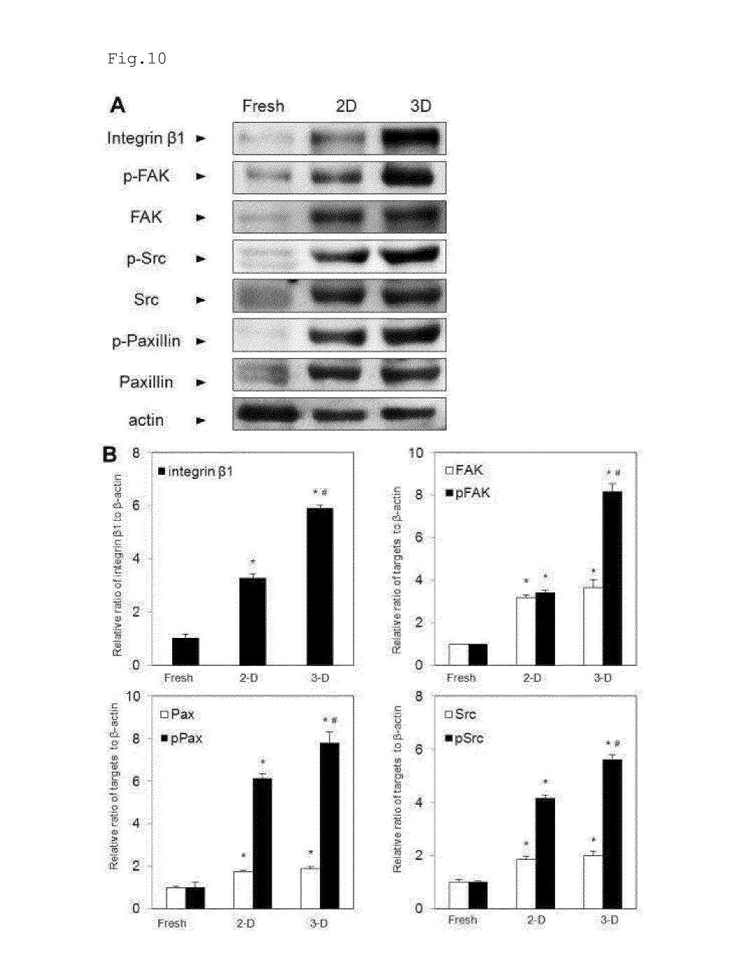

FIG. 10A shows Western blots of proteins involved in the integrin signaling pathway. Before organ culture, as shown in the Western blots, the integrin signaling pathway was not activated in the myocardial fragments before the culture (Fresh). Upon organ culture without a hydrogel support (2D), the integrin signaling pathway was activated to a significantly low degree, compared to the organ culture in an antifibrinolytic agent-containing hydrogel support (3D). This result was quantitatively confirmed as shown in FIG. 10B. The three-dimensional organ culture in a hydrogel support activated the integrin signaling pathway to a significantly higher degree than did the two-dimensional organ culture on a culture vessel. These results demonstrate that the three-dimensional, hydrogel-supported organ culture induces the activation of the integrin signaling pathway, thus stimulating the stem cells of cardiac muscles to grow.

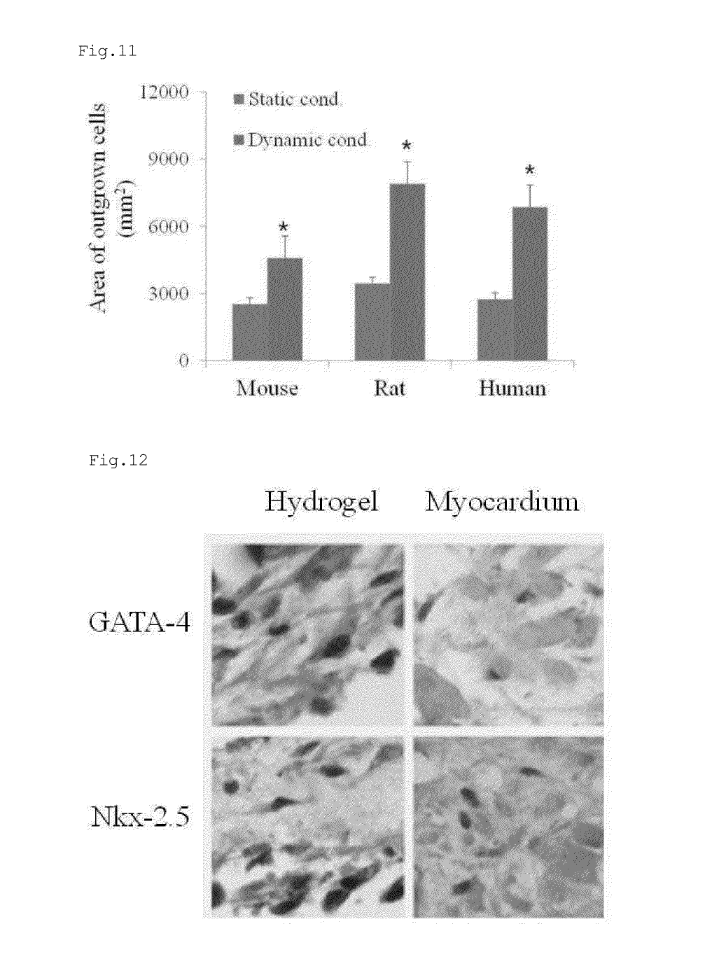

Comparison between cell growth in dynamic and static conditions is given in FIG. 11. As can be seen in the graph of FIG. 11, a dynamic condition promoted the supply of oxygen and nutrients to the cells during the three-dimensional hydrogel-supported organ culture, thereby increasing the area of the cells that grew in the hydrogel by 30% or greater, compared to a static condition (p<0.01).

### Example 8

### Properties of Cardiac Progenitor Cells that Grew Out of Myocardial fragments to Provisional Matrix-Mimic Hydrogel Containing Antifibrinolytic Agent

1) In Vitro BrdU-Labeling Analysis

Myocardial fragments were embedded into an antifibrinolytic agent-containing hydrogel in the same manner as in Example 8, and subjected to three-dimensional organ culture. From day 1 to day 3 of the organ culture, the myocardial fragments were incubated in the presence of 1 μM BrdU (Sigma). Paraffin blocks were prepared one week after the organ culture, and incubated with an anti-Nkx-2.5 (Abcam, Cambridge, Mass.) or anti-GATA-4 (Abcam) primary antibody and then with an isotype-matched Alexa Fluor 488-conjugated secondary antibody. Thereafter, the fragments were reacted with anti-BrdU (Sigma) and then with an isotype-matched Alexa Fluor 594-conjugated secondary antibody. Nuclei were strained with DAPI (Invitrogen) before confocal microscopy.

As shown in FIG. 12, the cells that had grown out of the fragments in the hydrogel expressed both the cardiomyocyte-specific transcription factors GATA-4 and Nkx-2.5. The cells expressing cardiomyocyte-specific transcription factors and stem cell markers are BrdU-positive, and were found to induce the growth of hydrocardiac progenitor cells capable of differentiating into cardiomyocytes on the three-dimensional, hydrogel-supported organ culture of myocardial fragments.

2) Immunological Traits of Cardiac Progenitor Cells Grown in Hydrogel

The paraffin blocks prepared for the in vitro BrdU-labeling analysis was used to examine the immunological properties of the cells which had grown out of the myocardial fragments in the antifibrinolytic agent-containing hydrogel. As primary antibodies for immunohistochemical staining, the cardiac progenitor cell marker nestin, the mesenchymal stem cell marker CD105, the vascular pericyte markers CD140b, CD146, and SMA, the hematopoietic cell marker CD34, and the vascular endothelial cell marker CD31 were employed.

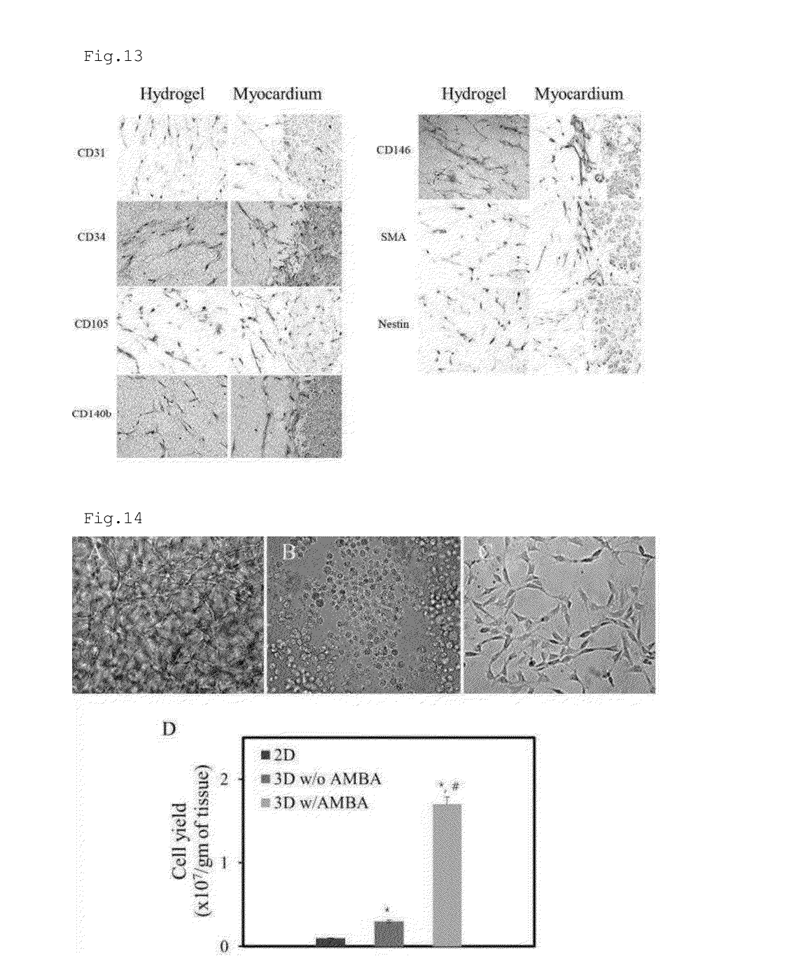

As can be seen in FIG. 13, the cells grown in the hydrogel had migrated from the interstitial stromal cells present between cardiomyocytes. All cells that grew in the hydrogel expressed markers specific for cardiac progenitor cells, mesenchymal stem cells, and vascular pericytes, but were negative to markers specific for hematopoietic cells and vascular endothelial cells.

### Example 9

### Recovery and Amplification of Cardiac Progenitor Cells Grown in Hydrogel

Myocardial fragments were cultured for one week in the same manner as in Example 7. The culture vessel was washed three times for 10 min at room temperature with phosphate-buffered saline (PBS) and then once with DMEM supplemented with 20% fetal bovine serum. To the culture vessel, 20 ml of DMEM supplemented with 20% fetal bovine serum and 10,000 units of urokinase (Green Cross, Seoul, Korea) was added. The culture vessel was shaken at 37° C. and 15 rpm for 30 min on an orbital shaker. When the antifibrinolytic agent-containing hydrogel was loosened and degraded, the cardiac progenitor cells and the myocardial fragments were transferred from the culture vessel to a 50 ml conical tube using a transfer pipette. Following centrifugation at 200×g for 10 min, the supernatant was discarded, and the remainder was added with 10 ml of a cell culture medium, and suspended using a pipette. The cardiac progenitor cells were separated from the myocardial fragments using a cell strainer with a diameter of 100 mm (BD Bioscience, Seoul, Korea). The recovered cells were assayed with a PicoGreen dsDNA Quantitation Kit, and the measurements were normalized to the weight of the myocardial fragments used in organ culture. Separately, after the enzymatic degradation of the myocardial fragments, cells were recovered and counted as illustrated above. Following centrifugation at 200×g for 10 min, the cell pellet was suspended in a medium. The cardiac progenitor cells in suspension were seeded at a density of 1×10⁴ cells/cm² into a culture vessel and amplified in a monolayer manner.

As can be seen in FIG. 14, the cardiac progenitor cells which grew in the antifibrinolytic agent-containing hydrogel appeared in a spindle shape and were distributed three-dimensionally. When the cell adhesion matrix was degraded by treatment with urokinase for 30 min, the cardiac progenitor cells took a round shape due to cytoplasmic shrinkage, and were distributed separately (FIG. 14B). These cells were observed to adhere in a monolayer pattern to a culture vessel within 30 min after seeding (FIG. 14C). The three-dimensional organ culture left for 7 days in an antifibrinolytic agent-containing hydrogel support (3D w/ AMBA) allowed production of 1.7×10⁷ cardiac progenitor cells from 1 mg of the myocardial fragment, showing 20-fold and 10-fold higher yields, compared to the two-dimensional culture following tissue dissociation, and the three-dimensional organ culture in an antifibrolytic agent-void hydrogel (3D w/o AMBA), respectively (p<0.01).

### Example 10

### Growth Characteristics of Cardiac Progenitor Cells Recovered from Hydrogel in Monolayer Culture Condition

The recovered cardiac progenitor cells were assayed for in vitro amplification performance in terms of colony forming unit-fibroblast (CFU-F) and population doubling time (PDT). CFU-F was determined by measuring the number of colonies formed after the cells recovered from the hydrogel were seeded at a density of 5 cells/cm² into 60 mm culture dishes and incubated for 11 days in a growth medium. For the measurement of PDT, the cardiac progenitor cells were seeded at a density of 2×10³ cell/well into 48-multiwell plates. On day 1 and 5 after incubation, the cells were lysed with CelLytic™ (Sigma). The DNA content of the cell lysate was measured using a PicoGreen dsDNA Quantitation Kit. Fluorescence intensity was measured at an emission wavelength of 485 nm and an excitation wavelength of 540 nm on a fluorescent microplate reader (Synergy™ HT; Bio-Tek Instruments, Neufahrn, Germany). Measurements of the DNA content were converted into cell counts using a standard curve. PDT was calculated according to the following formula: PDT=[(days in exponential phase)/((log N2−log N1)/log 2)] wherein N1 is the number of cells in an initial stage of the exponential phase and N2 is the number of cells in an end stage of the exponential phase.

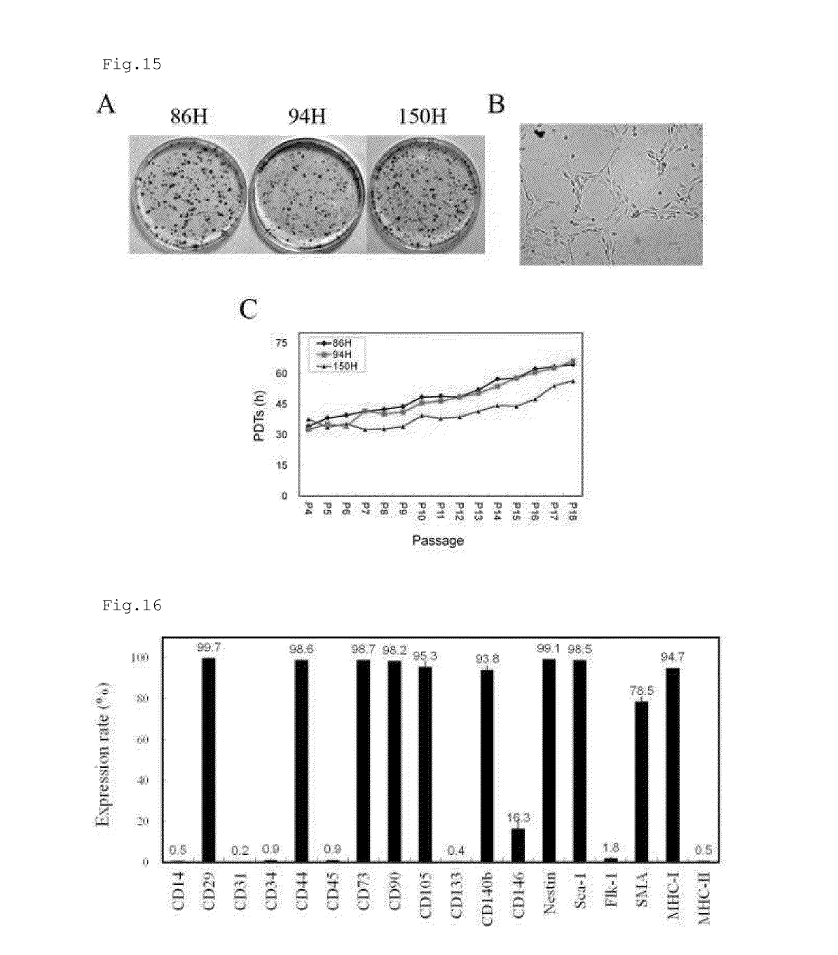

As shown in FIG. 15, the cardiac progenitor cells recovered from the hydrogel appeared in a spindle shape in a monolayer culture condition (FIG. 15B). Approximately 70% of the cardiac progenitor cells formed CFU-F (FIG. 15A). The cardiac progenitor cells were observed to be subcultured at least 20 times and to undergo at least 200 rounds of cell division. In addition, their PDT was measured to be 30 to 60 hrs, demonstrating their excellent cell division activity in a monolayer culture condition (FIG. 15C).

### Example 11

### Immunological Characteristics of Human Cardiac Progenitor Cells Recovered from Hydrogel

1) Immunological Characterization of Cardiac Progenitor Cells by Flow Cytometry

The cardiac progenitor cells that grew out of human myocardial fragment to an antifibrinolytic agent-containing hydrogel were amplified in a monolayer culture. Cells in a 3^{rd} passage were immunologically analyzed. In this regard, 1×10⁵ cells were incubated with fluorescent marker-conjugated human antibodies against CD14, CD29, CD31, CD34, CD73, CD90, and CD133. Non-conjugated antibodies against CD105 (R&D Systems), c-kit (DAKO, Glosrup, Denmark), Flk-1, PDGFR-β (CD140b; Abcam, Cambridge, Mass.), CD146 (Abcam), MHC (Abcam), SMA (DAKO), and nestin (Abcam) were reacted with a fluorescent marker-conjugated secondary antibody after application to 1×10⁵ cells for 30 min. Positivity to each antibody was analyzed in at least 10,000 cells using a flow cytometer (hereinafter referred to as “FCM”), manufactured by FACSCalibur (Becton Dickinson, San Jose, Calif.).

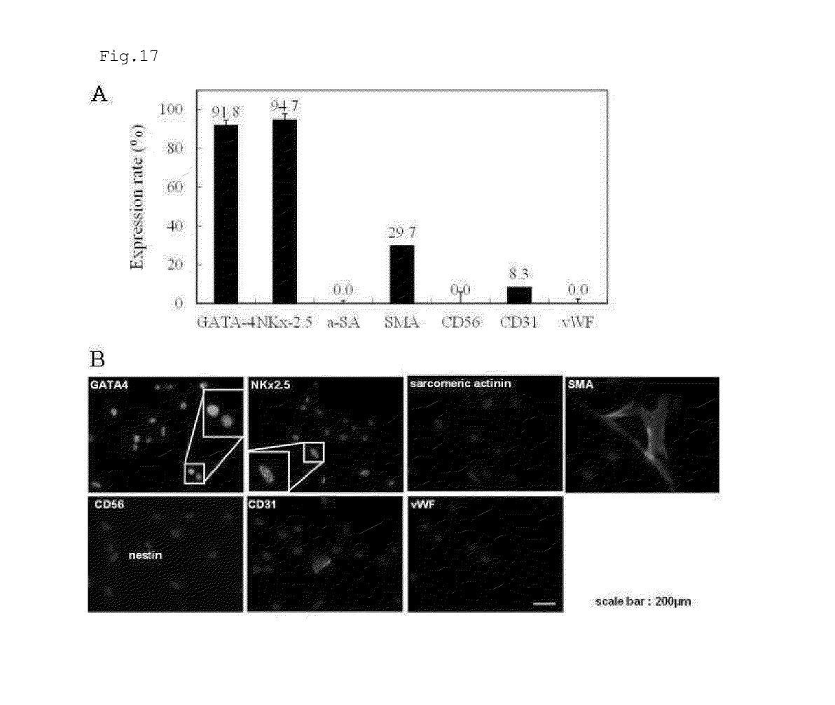

As is understood from the data of FIG. 16, more than 95% of the myocardium-resident cardiac progenitor cells were positive to the cardiac progenitor cell markers nestin and Sca-1, but negative to c-kit. The cardiac progenitor cells amplified by passages were positive to the mesenchymal stem cell markers CD29, CD44, CD73, CD90, and CD105, but negative to all of the hematopoietic cell markers CD14, CD34, CD45, c-kit, Flk-1, and CD133. More than 95% of the cardiac progenitor cells were observed to be positive to the MHC-I marker, but did not express the MHC-II marker, thus meeting the immunological condition necessary for allotransplantation. The vascular pericyte markers CD140b, CD146, and SMA were all expressed in cardiac progenitor cells, but at different frequencies thereamong.

2) Immunological Characterization by Immunofluorescence Staining

Using a cytocentrifuge (Cyto-Tek, Sakura, Tokyo, Japan), 1×10⁵ cardiac progenitor cells were attached to a glass slide. To detect the expression of the cardiomyocyte-specific transcription factors GATA-4 and Nkx-2.5, an immunofluorescence staining procedure was carried out in the same manner as in Example 8, followed by fluorescence microscopy. From at least 1,000 cells, fluorescence-positive cells were counted. In addition, an immunofluorescence staining examination was made of cells expressing the cardiomyocyte markers α-SA and CD56, the vascular endothelial cell markers CD31 and vWF, and the smooth muscle cell marker SMA.

As is understood from data of FIG. 17, the cardiomyocyte-specific transcription factors GATA-4 and Nkx-2.5 were expressed in more than 90% of the cardiac progenitor cells, demonstrating the potential of the cells to differentiate into cardiomyocytes. In contrast, α-SA, which is expressed in differentiated or mature cardiomyocytes, was not found in the cardiac progenitor cells. The cardiac progenitor cells amplified by passages were decreased in SMA expression level, and cells expressing SMA were estimated to have a potential to differentiate smooth muscle cells as they were morphologically similar to smooth muscle cells. In addition, the cardiac progenitor cells were evaluated to have a potential to differentiate into vascular endothelial cells as 8% of the cardiac progenitor cells were positive to CD31, a marker for immature vascular endothelial cells, but negative to vWF, a marker for mature vascular endothelial cells.

3) Immunological Properties of Nestin-Positive Cardiac Progenitor Cells

The cardiac progenitor cells on the 3^{rd} passage were stained in the same manner as in Example 8. The slides were incubated with an anti-nestin antibody and then with an isotype-matched Alexa Fluor 488-conjugated secondary antibody. Subsequently, the slides were reacted with anti-CD140b, anti-GATA-4, anti-Nkx-2.5, anti-α-SA, and anti-SMA antibodies, respectively, before incubation with an isotype-matched Alexa Fluor 594-conjugated secondary antibody. After staining nuclei with DAPI, the cells were observed under a confocal microscope. A total of 1000 nestin-positive cells were examined for positivity to the target proteins.

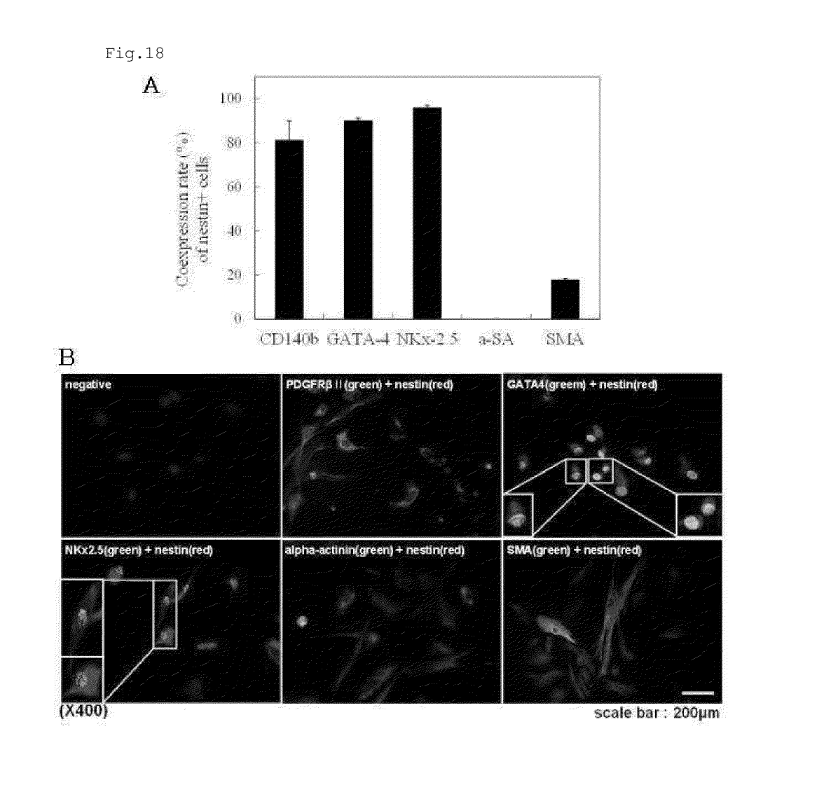

As can be seen in FIG. 18, nestin-positive cardiac progenitor cells expressed the vascular pericyte marker CD140b and the cardiomyocyte-specific transcription factors GATA-4 and Nkx-2.5 at a rate of 80%, but did not express the cardiomyocyte marker α-SA. The smooth muscle cell marker SMA was found in the cardiac progenitor cells.

### Example 12

### Differentiation of In Vitro Amplified Cardiac Progenitor Cells into Cardiomyocytes

1) Cardiosphere Formation

The cardiac progenitor cells amplified in a monolayer culture condition after recovery from the hydrogel were examined for the ability to form cardiospheres. The cardiac progenitor cells were seeded at a density of 2×10⁵ cells/well into 6-multiwell plates. A medium for inducing the formation of cardiospheres was comprised of 98% (vol/vol) DMEM, 2% (vol/vol) B27 (Invitrogen), 20 ng/ml EGF, and 20 ng/ml bFGF. After incubation for 3 days in the medium, cardiospheres were observed under a phase-contrast microscope. In addition, the cardiac progenitor cells with cardiospheres were examined for protein expression by immunofluorescence staining.

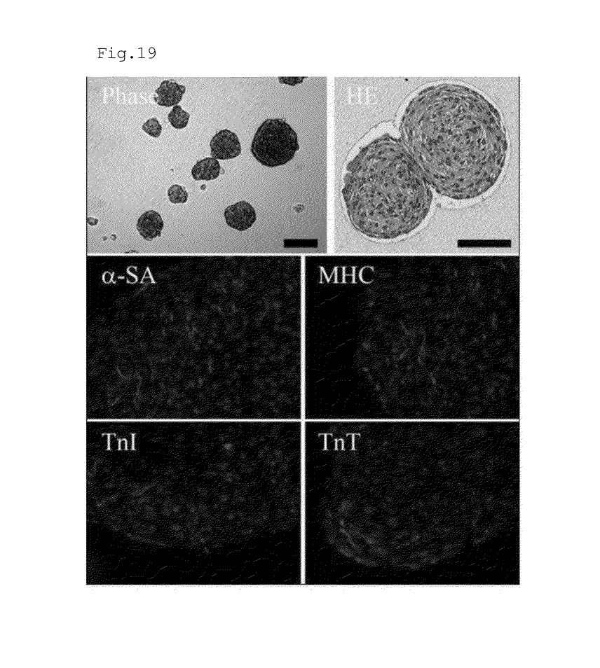

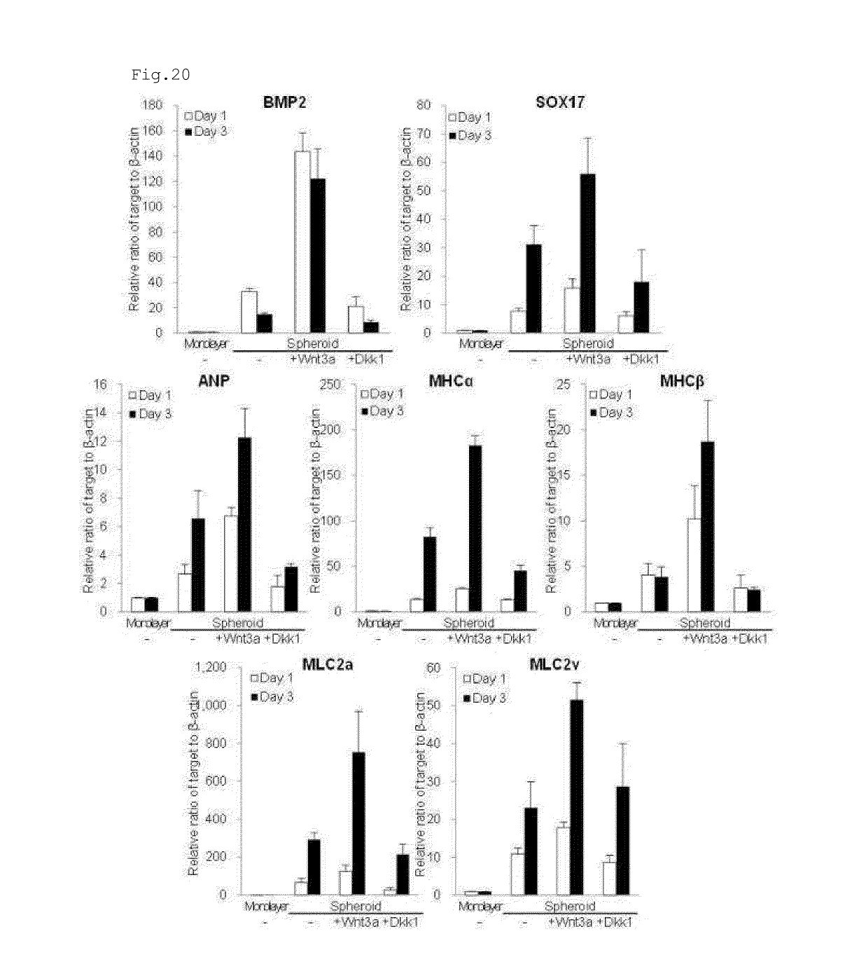

As visualized in FIG. 19, the cardiac progenitor cells started to form cardiospheres (CS) from 2 days after monolayer culture, but the cardiospheres did not increase in size with time. The cardiac progenitor cells with cardiospheres were positive to α-SA, MHC, TnI, and TnT markers found in mature cardiomyocytes.

2) Assay System for Differentiation into Cardiomyoctes Using Suspension Cell Culture

A suspension of cardiac progenitor cells in a medium inducing myocardial differentiation was seeded at a density of 20,000 to 100,000 cells/well into polymer-coated 24-multiwell tissue culture plates preventive of cell and protein adhesion (Ultra-Low Attachment Surface, Corning, Lowell, Mass.). The medium was supplemented with 1 mM Wnt3a or Dkk1 before culturing the cardiac progenitor cells in a suspension culture condition or a monolayer culture condition. After culturing for 1 and 3 days, RNA was isolated from the cardiac progenitor cells and used to synthesize cDNA. Real-time PCR was performed on this cDNA to quantify mRNA levels of bone morphogenetic protein 2 (BMP2), SOX17, ANP, α-MHC, β-MHC, MLC2a, and MLC2v (myosin light chain-2 ventricle), which are involved in the formation of cardiospheres or the Wnt signaling pathway, thus evaluating the potential to differentiate into cardiomyocytes.

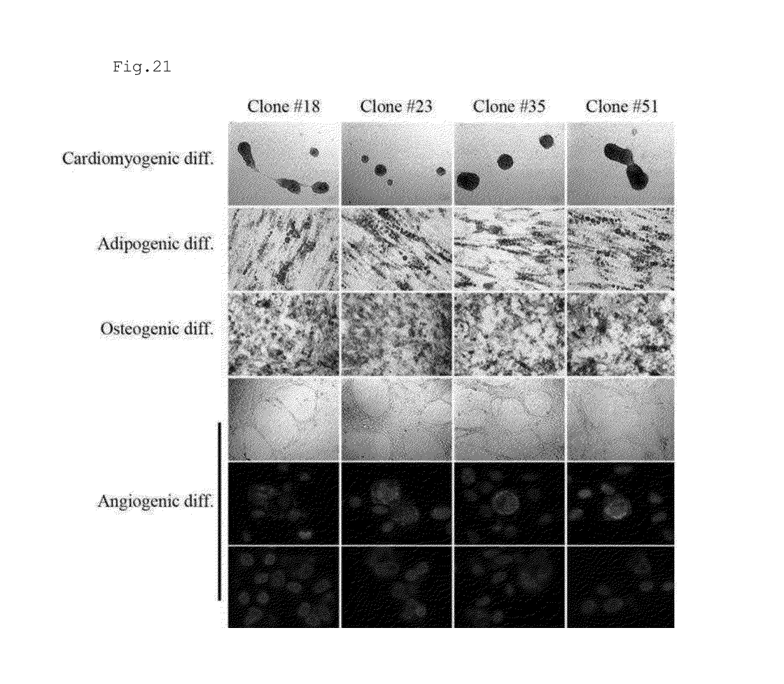

As is apparent from the data of FIG. 20, the suspension cell culture condition significantly increased the mRNA levels of BMP2 and Sox 17, which are factors inducing differentiation into cardiomyocytes, in the cardiac progenitor cells, compared to the monolayer culture condition. The mRNA levels of BMP2 and SOX17 were increased by Wnt3a, but decreased by Dkk1. In addition, the mature cardiomyocyte-specific markers arterial natriuretic peptide (ANP), α-MHC, β-MHC, MLC-2a (myosin light chain-2 atrium), and MLC2v (myosin light chain-2 ventricle) were significantly increased in mRNA level by suspension cell culture, compared to monolayer cell culture. Also, these markers' mRNA levels were increased by Wnt3a and reduced by Dkk1. Further, the cardiospheroic cells expressed the genes at higher mRNA levels on day 3 than day 1.

### Example 13

### In Vitro Pluripotency of Single Clone-Derived Cardiac Progenitor Cells

1) Isolation and Amplification of Single Clone-Derived Cardiac Progenitor Cells

Cardiac progenitor cells isolated from cardiac muscle tissues of 5 different donators were seeded at a density of 0.5 cells/well into 96-multiwell tissue culture plates containing a growth culture medium. After incubation for 24 hrs, wells in each of which only one single cell grew were selected. When the cell multiplied in number to form a cell aggregate, it was detached from the plate by trypsinization and transferred into 24-multiwell tissue culture plates. When the cells grew to 80˜90% confluence, they were transferred again into 6-multiwell tissue culture plates and amplified therein. The resulting monoclonal cardiac progenitor cells exhibited clone formation at a rate of 69.8±5.6% on average. Of them, at least 20 clones were selected and assayed for the potential to differentiate into cardiomyocytes, adipocytes, and vascular endothelial cells.

2) Differentiation into Cardiomyocytes

Using the suspension cell culture method explained in Example 12-2, the potential of the single cell-derived cardiac progenitor cells to differentiate into cardiomyocytes was evaluated. The cardiac progenitor cells were incubated for 8 days in a medium (98.9% DMEM, 1% CS, 0.1% DMSO, 50 μM ascorbic acid) designed to induce differentiation into cardiomyocytes. The differentiation of the cardiac progenitor cells into cardiomyocytes was evaluated in terms of cardiospheric formation.

As can be seen in FIG. 21, the single clone-derived cardiac progenitor cells formed cardiospheres and expressed cardiomyocyte-specific proteins, demonstrating their potential to differentiate into cardiomyocytes. Cardiospheric formation indicative of myocardial differentiation was observed in 72% of the single clone-derived cardiac progenitor cells.

3) Differentiation into Adipocytes

The potential of the single clone-derived cardiac progenitor cells to differentiate into adipocytes was evaluated. In this regard, 200,000 cells were seeded into 24-multiwell tissue culture plates and cultured for 14 days in a medium comprising 90% DMEM, % CS, 0.5 mM 3-isobutyl-1-methylxanthine (Sigma), 80 μM indomethacin (Sigma), 1 μM dexamethasone (Sigma), and 5 μg/ml insulin (Sigma). The differentiation of the single clone-derived cardiac progenitor cells into adipocytes was evaluated by examining the cytoplasmic accumulation of lipid. The result is given in FIG. 21.

The single clone-derived cardiac progenitor cells were found to have the potential to differentiate into adipocyte, as visualized after staining at room temperature for 1 hr with 0.5% Oil Red O (Sigma).

4) Differentiation into Vascular Endothelial Cells

The single clone-derived cardiac progenitor cells were induced to differentiate into vascular endothelial cells. For this, 200,000 single clone-derived cardiac progenitor cells were seeded into 300 μl of a hydrogel containing 2.5 mg/ml fibrinogen and 0.5 U/ml thrombin. Differentiation into vascular endothelial cells was carried out by incubation for 7 days in a medium comprising 98.5% DMEM, 1% CS, 0.5% DMSO, 10 ng/ml VEGF (R&D systems), 10 ng/ml EGF, and 10 ng/ml bFGF. The differentiation was determined by examining the formation of capillary vessel-like net structures and the expression of markers specific for vascular endothelial cells.

As can be seen in FIG. 21, all of the single clone-derived cardiac progenitor cells formed capillary vessel-like net structures and were positive to CD31, a marker for vascular endothelial cells.

### Example 14

### Secretion Properties of In Vitro Amplified Cardiac Progenitor Cells

One million cardiac progenitor cells or muscle stem cells were seeded into a 100-mm culture dish containing DMEM supplemented with 1% fetal bovine serum and cultured for 1 day. Then, only the culture medium was collected and centrifuged at 100×g. The supernatant was filtered. Separately, an antibody array membrane was blocked at room temperature for 30 min with a blocking buffer. The antibody array membrane was incubated in the filtered supernatant at 4° C. for 16 hrs and washed three times with a washing buffer. Subsequently, the antibody array membrane was reacted at room temperature for 1 hr with a biotin-conjugated antibody, washed with a washing buffer, and incubated at room temperature for 2 hrs with horseradish peroxidase (HRP)-conjugated streptavidin. Color development was performed with a detection buffer, and images were taken by an LAS3000 system. Expression levels of angiogenesis factors were compared between the cardiac progenitor cells and the muscle stem cells using the signal intensity MultiGauge v2.2 program.

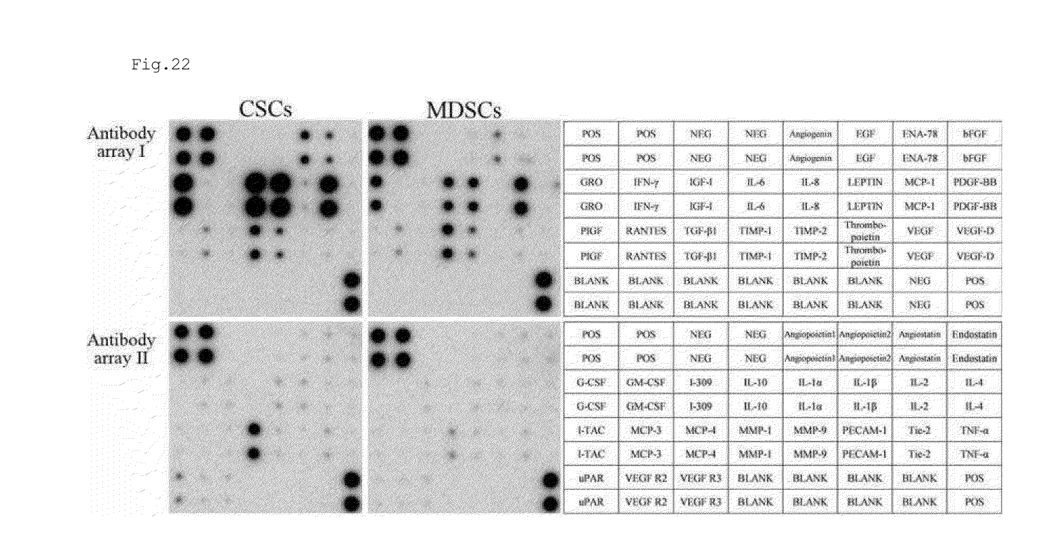

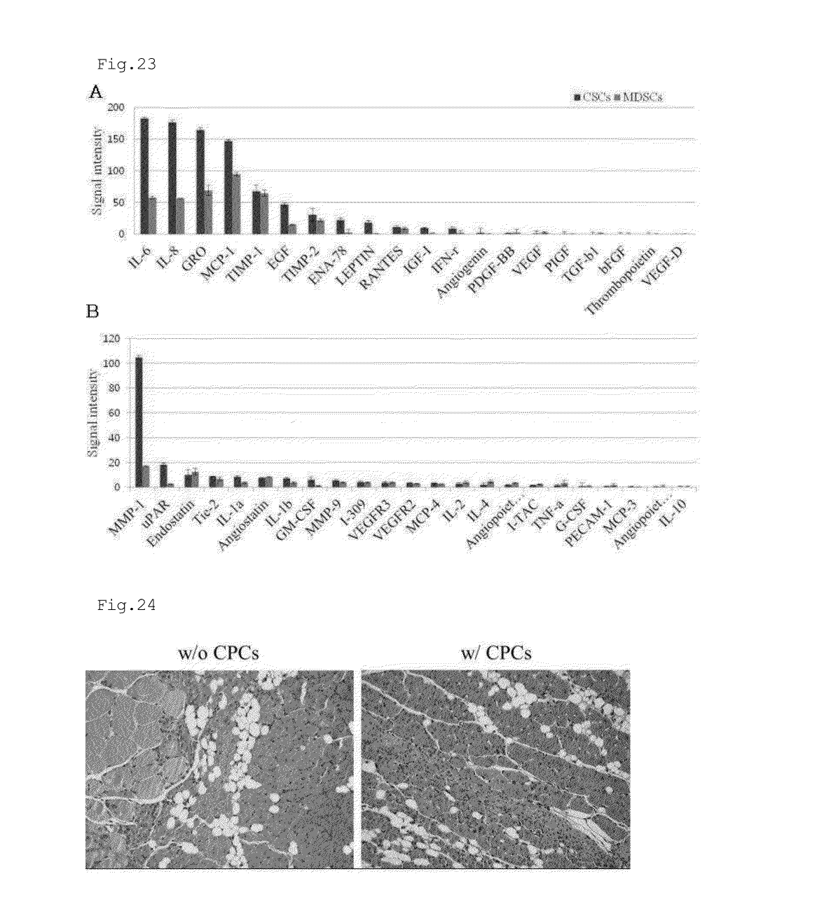

FIG. 22 shows human antibody arrays indicating angiogenesis factors secreted from the cardiac progenitor cells or muscle stem cells, together with relevant tables. FIG. 23 shows graphs in which proteins secreted from cardiac progenitor cells and skeletal muscle-derived stem cells are quantitatively plotted. In this experiment, a total of 44 tissue regeneration-related factors were analyzed. The cardiac progenitor cells secreted more various factors, compared to the muscle stem cells. Of the factors, leptin, insulin-like growth factor I (IGF-I), placenta growth factor (PIGF), epithelial neutrophil-activating peptide-78 (ENA-78), urokinase plasminogen activator receptor (uPAR), matrix metalloproteinase-1 (MMP-1), granulocyte-macrophage colony-stimulating factor (GM-CSF), interferon gamma (IFN-γ), interleukin-6 (IL-6), interleutin-8 (IL-8), interleutkin-1α (IL-1α), epidermal growth factor (GM-CSF), and growth-regulated protein (GRO) were found to be secreted at a 2-fold higher level from the cardiac progenitor cells than from the muscle stem cells.

### Example 15

### Revascularization Ability of In Vitro Amplified Cardiac Progenitor Cells

In order to verify the in vivo vascularization ability of the cardiac progenitor cells, murine models of hindlimb ischemia were employed. Hindlimb ischemia was caused in male BALB/c mice, 9˜10 weeks old, by ligation of the femoral artery. The next day, two million cardiac progenitor cells labeled with CM-DiI were induced into a hindlimb muscle. Revascularization of the injected cardiac progenitor cells was evaluated by laser Doppler-based perfusion measurements. Ischemic damage of the hindlimb muscle was analyzed by hematoxylin-eosin staining. To quantitate the density of microvessels per area of the hindlimb muscle, immunofluorescence staining was conducted for CD34, a marker specific for vascular endothelial cells.

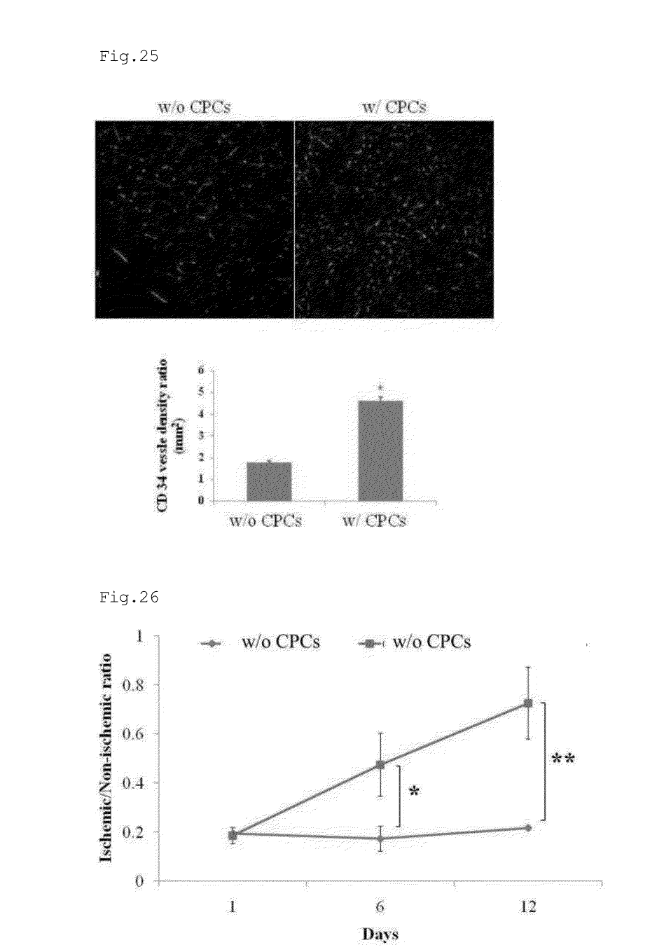

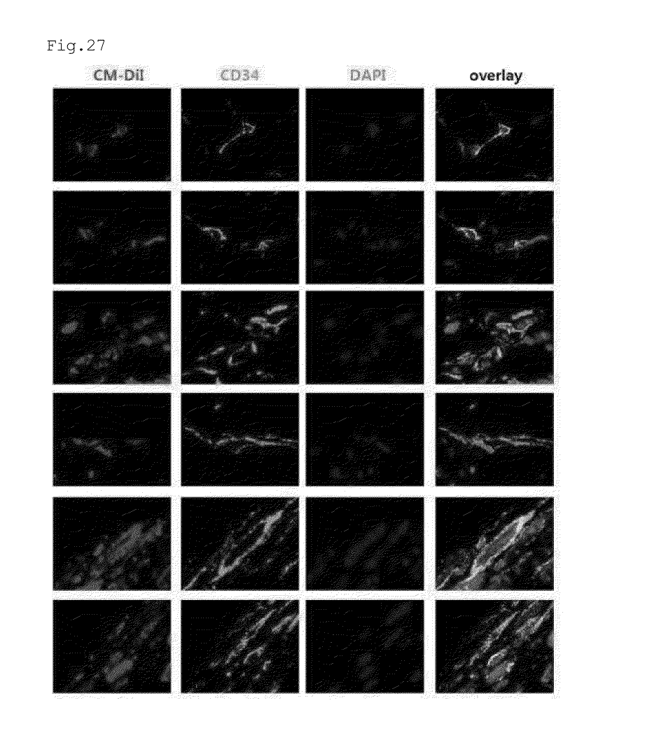

FIG. 24 shows photographs of hindlimb muscle tissues of mice into which hindlimb ischemia was induced. When not treated with the cardiac progenitor cells (w/o CPCs), the mice suffered from significant necrosis in the hindlimb muscle (left panel). In contrast, injection of the cardiac progenitor cells into the hindlimb muscle (w/CPCs) reduced the ischemic damage and increased the regeneration of skeletal muscles (right panel). FIG. 25 shows densities of CD34-positive microvessels in the ischemic hindlimb muscle. A higher density of CD34-positive microvessels was found in the ischemic hindlimb muscles injected with the cardiac progenitor cells (right photograph) than in the non-treated ischemic hindlimb muscles (left photograph). The density of CD34-positive microvessels in the cardiac progenitor cell-injected group was twice that in the non-treated group (*, p<0.01). As is understood from the data of FIG. 26, there were no differences in the blood flow rate of the sole of the foot one day after the ligation of the femoral artery whether the cardiac progenitor cells were injected or not. On day 5 and day 11, however, the mice injected with the cardiac progenitor cells were significantly increased in blood flow rate, compared to non-treated mice. FIG. 27 shows photographs of CM-DiI-labeled cardiac progenitor cells traced with time in murine models of hindlimb ischemia after injection of two million CM-DiI-labeled cardiac progenitor cells to the models, illustrating the role and the differentiation properties of the injected cardiac progenitor cells in vivo. As seen in FIG. 27, the injected cardiac progenitor cells were observed to differentiate into CD34-positive vascular endothelial cells involved in the formation of microvessels. Since CM-DiI signals were coincident with CD34 signals, most of the injected cardiac progenitor cells were differentiated into vascular endothelial cells in the ischemic hindlimb model.

### Example 16

### Delivery of Cardiac Progenitor Cells to the Heart Using Antifibrinolytic Agent-Containing Hydrogel

Human cardiac progenitor cells were labeled with CM-DiI before transplantation into cardiac muscles. Sprague-Dawley rats received ligation of the proximal left anterior descending coronary artery to induce acute myocardial infarction. Separately, two million human cardiac progenitor cells labeled with CM-DiI were suspended in a 5 mg/ml fibrinogen solution, and the suspension was mixed with one volume of a 1 unit/ml thrombin solution. Immediately after the ligation, the mixture was injected into cardiac muscles using a 1 ml syringe. The mixture was immediately formed into a gel when injected. For a control, CM-DiI-labeled human cardiac progenitor cells were suspended in physiological saline and injected to rats. To assess the effect of the hydrogel on the delivery of cardiac progenitor cells to cardiac muscles, the heart was excised one day after injection. The excised heart was sectioned in a thickness of mm, after which CM-DiI signal intensity was measured using a fluorescence scanner (Typhoon, Amersham, UK). The distribution area of the cells injected to the cardiac muscles was determined with the aid of the Image J program.

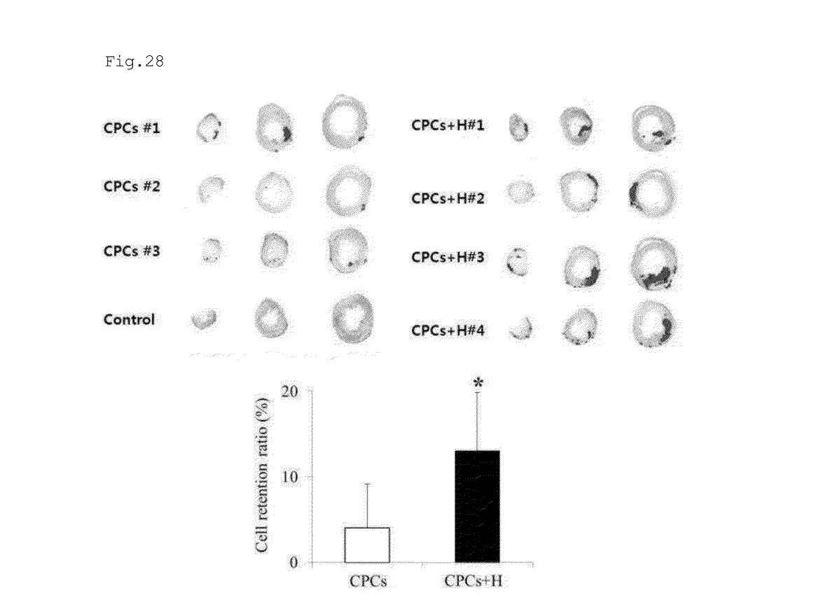

As shown in FIG. 28, human cardiac progenitor cells occupied 5% of the area of the cardiac muscles when injected alone, but were distributed over more than 10% of the area when delivered by the antifibrinolytic agent-containing hydrogel (CPCs+H).

### Example 17

### Myocardial Regeneration of Cardiac Progenitor Cells

An acute myocardial infarction was induced in Sprague Dawley rats by ligation of the left anterior coronary artery as in Example 16. After the occurrence of edema and necrosis in the myocardium, two million human cardiac progenitor cells were suspended in physiological saline or embedded into hydrogel before injection into the myocardial (refer to Example 17). To overcome the immune rejection against human cardiac progenitor cells, an immunosuppressive agent (100 mg/kg, cyclosporin) was injected every day. Two weeks after transplantation of human cardiac progenitor cells, the heart was excised, and used to prepare paraffin blocks. Collagen staining was performed on the paraffin blocks, the thickness of the left ventricle was determined using the Image J program, and the fibrotic area of the myocardium was calculated. Immunofluorescence staining with an SMA antibody was carried out to assess the density of microvessels in the heart. The tissue regeneration and differentiation properties of the cardiac progenitor cells injected into the myocardium were monitored by double immunofluorescence staining. The human cardiac progenitor cells injected into the myocardium were labeled with an anti-human mitochondria antigen (HMA), together with troponin I (TNI) for monitoring differentiation into cardiomyocytes, SMA for monitoring smooth muscle cells, or isolectin B4 (Isolectin) for monitoring vascular endothelial cells. Signal detection was made by means of an Alexa Fluor-594-conjugated secondary antibody for HMA and by means of an Alexa Fluor-488-conjugated secondary antibody for TNI, SMA, or Isolectin.

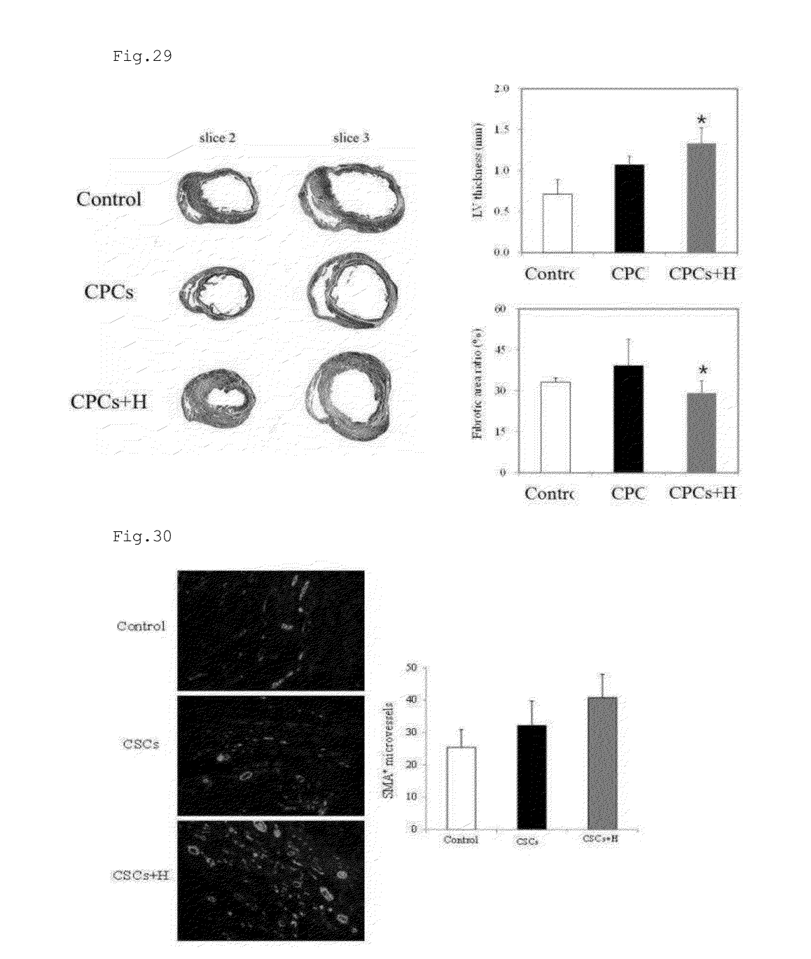

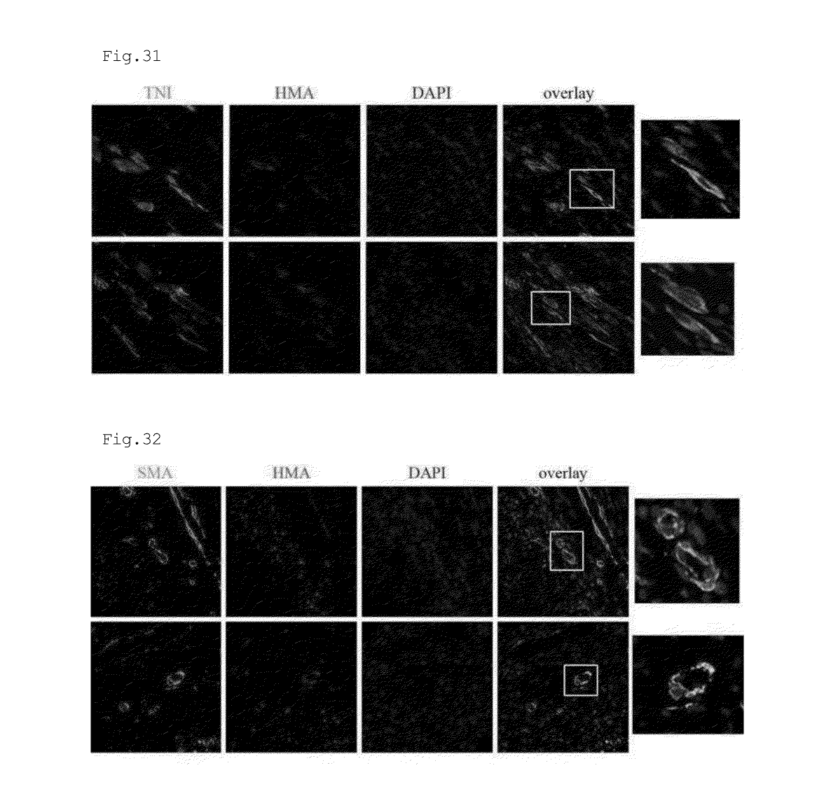

As can be seen in FIG. 29, the rats injected with the cardiac progenitor cells embedded into an antifibrinolytic agent-containing hydrogel (CPCs+H) had significantly reduced myocardial damage, with significant regeneration of the myocardium, compared to the rats injected with physiological saline only (control) or with a suspension of the cardiac progenitor cells in physiological saline (CPCs). The highest wall thickness of the left ventricle (LV thickness) was found in the rats transplanted with the cardiac progenitor cells embedded into a hydrogel. In contrast, the rats injected with physiological saline or cardiac progenitor cells alone had a reduced wall thickness of the left ventricle, with distension of the left atrium. In addition, post-myocardial infarction fibrosis (fibrotic area) was significantly reduced in the heart injected with the cardiac progenitor cells embedded into an antifibrinolytic agent-containing wound matrix hydrogel.

As is understood from data of FIG. 30, the density of myocardial microvessels was the lowest in the heart injected with physiological saline alone (control) and the highest in the heart injected with the cardiac progenitor cells embedded into an antifibrinolytic agent-containing hydrogel (CSCs+H).

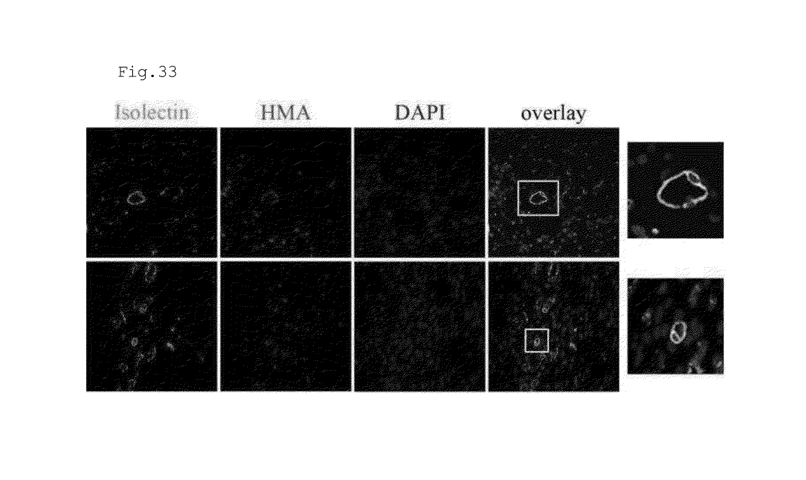

In the fluorescence photographs of FIG. 32, TNI, a characteristic of cardiomyocytes, is detected in the human cardiac progenitor cells labeled with HMA (red), indicating the differentiation of the human cardiac progenitor cells into cardiomyocytes. Also, the human cardiac progenitor cells were differentiated into vascular smooth muscle cells as demonstrated by the positional coincidence of HMA signals (red) with SMA signals (green). As can be seen in FIG. 33, the HMA-positive human cardiac progenitor cells (red) took a tubular structure, with the concomitant expression of isolectin (green), indicating that the injected human cardiac progenitor calls differentiated into vascular endothelial cells to form vessels.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. A method of culturing myocardium-resident cardiac progenitor cells, comprising:
embedding myocardial fragments into hydrogel;
culturing the myocardial fragment embedded into hydrogel;
degrading only the hydrogel to recover cardiac progenitor cells grown out of the myocardial fragment to the hydrogel; and
amplifying the cardiac progenitor cells in vitro.

2. The method of claim 1, wherein the cardiac progenitor cells exhibit at least one immunological trait of:
(i) being positive to a cardiac progenitor cell marker selected from the group consisting of nestin, Sca-1, and a combination thereof;
(ii) being positive to cardiomyocyte-specific transcription factor marker selected from the group consisting of GATA-4, Nkx-2.5, Mef-2c, and a combination thereof;
(iii) being positive to a mesenchymal stem cell marker selected from the group consisting of CD29, CD44, CD73, CD90, CD105, and a combination thereof;
(iv) being positive to a vascular pericyte marker selected from the group consisting of CD140b, CD146, α-smooth muscle actin (SMA), and a combination thereof;
(v) being negative to a hematopoietic cell marker selected from Lin, CD34, CD45, and a combination thereof;
(vi) being negative to a vascular endothelial cell marker selected from the group consisting of CD31, CD34, and a combination thereof; and
(vii) being negative to a cardiomyocyte marker selected from the group consisting of α-sarcometric actinin (α-SA), myosin heavy chain (MHC), troponin I (TnI), troponin T (TnT), and a combination thereof.

3. The method of claim 1, wherein the hydrogel is made of a natural polymer.

4. The method of claim 1, wherein the hydrogel contains an antifibrinolytic agent.

5. The method of claim 4, wherein the antifibrinolytic agent is selected from the group consisting of aminocaproid acid, tranexamic acid, aprotinin, aminomethylbenzoic acid, and a combination thereof.

6. The method of claim 4, wherein the antifibrinolytic agent is contained in an amount of from 10 to 1000 μg per ml of the hydrogel

7. The method of claim 1, wherein the hydrogel is a fibrin hydrogel, and contains fibrinogen in a concentration of from 0.8 to 5.0 mg/ml.

8. The method of claim 1, wherein the hydrogel is degraded by an enzyme selected from the group consisting of collagenase, gelatinase, urokinase, streptokinase, tissue plasminogen activator (TPA), plasmin, hyaluronidase, and a combination thereof.

9. The method of claim 1, wherein:
1) the embedding is carried out by mixing the myocardial fragment with a fibrin hydrogel containing an antifibrinolytic agent selected from the group consisting of aminocaproid acid, tranexamic acid, and a combination thereof;
2) the culturing is carried out by subjecting the myocardial fragment embedded into the fibrin hydrogel to three-dimensional organ culture while shaking at 5 to 30 rpm to allow myocardial-resident cardiac progenitor cells to grow out of the myocardial fragment to the hydrogel;
3) the degrading is carried out by treating the fibrin hydrogel with an enzyme selected from the group consisting of urokinase, streptokinase, plasmin, and a combination thereof to recover the myocardium-resident cardiac progenitor cells, and the myocardial fragment; and
4) the amplifying is carried out by culturing the cardiac progenitor cells recovered from the hydrogel in a monolayer culture condition.

10. The method of claim 9, wherein the recovered myocardial fragment is recycled by being embedded again into a hydrogel.

11. Cardiac progenitor cells, obtained using the culturing method of claim 1, exhibiting at least one immunological trait of:
(i) being positive to a cardiac progenitor cell marker selected from the group consisting of nestin, Sca-1, and a combination thereof;
(ii) being positive to a cardiomyocyte-specific transcription factor marker selected from the group consisting of GATA-4, Nkx-2.5, Mef-2c, and a combination thereof;
(iii) being positive to a mesenchymal stem cell marker selected from the group consisting of CD29, CD44, CD73, CD90, CD105, and a combination thereof;
(iv) being positive to a vascular pericyte marker selected from the group consisting of CD140b, CD146, SMA, and a combination thereof;
(v) being negative to a hematopoietic cell marker selected from Lin, CD34, CD45, and a combination thereof;
(vi) being negative to a vascular endothelial cell marker selected from the group consisting of CD31, CD34, and a combination thereof; and
(vii) being negative to a cardiomyocyte marker selected from the group consisting of α-sarcometric actinin (α-SA), myosin heavy chain (MHC), troponin I (TnI), troponin T (TnT), and a combination thereof.

12. The cardiac progenitor cells of claim 11, having a potential to differentiate into cardiomyocytes, osteoblasts, adipocytes, chondrocytes, vascular endothelial cells, smooth muscle cells, neural cells, or skeletal muscle cells.

13. A method for differentiating myocardium-resident cardiac progenitor cells, comprising culturing the cardiac progenitor cells of claim 11 in a suspension cell culture condition.

14. The method of claim 13, wherein the cardiac progenitor cells are capable of spontaneously differentiate into cardiomyocytes.

15. A cell therapeutic agent, comprising the cardiac progenitor cells of claim 11, or cells differentiated therefrom, as an active ingredient.

16. The cell therapeutic agent of claim 15, treating cells' selected from the group consisting of cardiomyocytes, osteoblasts, adipocytes, chondrocytes, vascular endothelial cells, smooth muscle cells, neural cells, skeletal muscle cells, and a combination thereof.

17. The cell therapeutic agent of claim 15, wherein the cardiac progenitor cells are in mixture with a hydrogel containing an antifibrinolytic agent.

18. The cell therapeutic agent of claim 15, further comprising a factor selected from the group consisting of an anti-inflammatory agent, a stem cell-mobilizing factor, a vascular growth inducing factor, and a combination thereof.

19. A pharmaceutical composition for prophylaxis or therapy of a heart disease, comprising the cardiac progenitor cells of claim 11 or cells differentiated therefrom as an active ingredient, wherein the progenitor cells are in mixture with a hydrogel containing an antifibrolytic agent.

20. The pharmaceutical composition of claim 19, wherein the heart disease is selected from the group consisting of myocardial infarction, ischemic myocardial disease, primary myocardial disease, secondary myocardial disease, congestive heart failure, and a combination thereof.
